(19) United States
(12) Patent Application Publication
(10) Pub. No.: US 2024/0374789 A1
(43) Pub. Date: Nov. 14, 2024

(54) TISSUE REPAIR MATERIAL KIT AND TISSUE REPAIR METHOD

(71) Applicant: FUJIFILM Corporation, Tokyo (JP)

(72) Inventors: Kazuto FUKUNAGA, Kanagawa (JP); Nobuhiko KATO, Kanagawa (JP)

(73) Assignee: FUJIFILM Corporation, Tokyo (JP)

(21) Appl. No.: 18/612,129

(22) Filed: Mar. 21, 2024

Related U.S. Application Data
(63) Continuation of application No. PCT/JP2022/029561, filed on Aug. 1, 2022.

(30) Foreign Application Priority Data
Sep. 30, 2021 (JP) 2021-162131

(51) Int. Cl. A61L 27/22; A61K 38/39; C07K 14/78

(52) U.S. Cl. CPC A61L 27/222; A61K 38/39; C07K 14/78; A61L 2400/06; A61L 2430/02

(57) ABSTRACT

The application provides a tissue repair material kit and a tissue repair method, where the tissue repair material kit includes a cylindrical container that has an opening portion having an opening diameter smaller than an inner diameter of the container and a tissue repair material, in which in a case where the tissue repair material is supplied, a storage clastic modulus of the tissue repair material at 25° C. is 0.1 kPa to 100 kPa.

### CROSS-REFERENCE TO RELATED APPLICATION

This application is a Continuation of International Application No. PCT/JP2022/029561, filed Aug. 1, 2022, which claims priority to Japanese Patent Application No. 2021-162131, filed Sep. 30, 2021. Each of the above applications is hereby expressly incorporated by reference, in its entirety, into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 29, 2024, is named “1982-1374PUS1.xml” and is 502,530 bytes in size. The sequence listing contained in this .XML file is part of the specification and is hereby incorporated by reference herein in its entirety.

### BACKGROUND OF THE INVENTION

### 1. Field of the Invention

The present disclosure relates to a tissue repair material kit and a tissue repair method.

### 2. Description of the Related Art

A tissue repair material is used to form tissue (for example, a bone) in a tissue-deficient part (for example, a bone-deficient part) caused by, for example, injuries, surgery, or the like. A bone tissue repair material, which is one of such tissue repair materials, is disclosed in, for example, JP2018-094401A.

### SUMMARY OF THE INVENTION

Techniques related to tissue repair, including the technique of JP2018-094401A, have been studied in the related art. However, at present, a technique for supplying a tissue repair material to a narrow tissue-deficient part is not sufficient.

The present disclosure has been made in consideration of such circumstances, and an object to be achieved by an embodiment of the present disclosure is to provide a tissue repair material kit that makes it possible to easily supply a tissue repair material to a narrow tissue-deficient part.

An object to be achieved by another embodiment of the present disclosure is to provide a tissue repair method using the tissue repair material kit.

The present disclosure includes the following aspects.

<1>A tissue repair material kit comprising:
a cylindrical container that has an opening portion having an opening diameter smaller than an inner diameter of the container: and
a tissue repair material,
in which in a case where the tissue repair material is supplied, a storage elastic modulus of the tissue repair material at 25° C. is 0.1 kPa to 100 kPa.

<2>The tissue repair material kit according to <1>, further comprising a discharge part that has an inner diameter smaller than the inner diameter of the cylindrical container and is provided at the opening portion.

<3>The tissue repair material kit according to <1>or <2>, in which the opening diameter of the opening portion or an inner diameter of an discharge part is 10% to 70% of the inner diameter of the cylindrical container.

<4>The tissue repair material kit according to any one of <1>to <3>, in which the opening diameter of the opening portion or an inner diameter of an discharge part is 5 mm or less.

<5>The tissue repair material kit according to any one of <1>to <4>, in which the tissue repair material has a particulate shape in a dry state, and the opening diameter of the opening portion or an inner diameter of an discharge part is 40% to 300% of a maximum particle diameter of the tissue repair material in the dry state.

<6>The tissue repair material kit according to any one of <1>to <5>, further comprising:
a pressurization unit,
wherein the opening portion is provided at one end of the cylindrical container, and
the pressurization is provided at the other end of the cylindrical container.

<7>The tissue repair material kit according to <6>, in which the pressurization unit is a plunger.

<8>The tissue repair material kit according to any one of <1>to <7>, in which the tissue repair material is accommodated in the cylindrical container.

<9>The tissue repair material kit according to any one of <1>to <8>, in which a water absorption rate of the tissue repair material is 300% to 2,000%.

<10>The tissue repair material kit according to any one of <1>to <9>, in which in a case where the tissue repair material contains a water-containing liquid, the storage elastic modulus of the tissue repair material at 25° C. is 0.1 kPa to 100 kPa in a case where a value obtained by multiplying a mass fraction of a content of the water-containing liquid with respect to a dry mass of the tissue repair material by a tap density of the tissue repair material in a dry state is 0.40 g/mL to 0.70 g/mL.

<11>The tissue repair material kit according to any one of <1>to <10>, further comprising a water-containing liquid for mixing with the tissue repair material.

<12>The tissue repair material kit according to <11>, in which a content of the water-containing liquid is 50% by mass to 2,000% by mass with respect to a dry mass of the tissue repair material.

<13>The tissue repair material kit according to any one of <1>to <12>, in which the tissue repair material contains a biocompatible polymer.

<14>The tissue repair material kit according to <13>, in which the biocompatible polymer is a recombinant peptide.

<15>The tissue repair material kit according to <14>, in which the recombinant peptide includes the following peptide of (A), (B), or (C),
(A) a peptide consisting of an amino acid sequence set forth in SEQ ID NO: 1
(B) a peptide consisting of an amino acid sequence, in which one or several amino acid residues in the amino acid sequence set forth in SEQ ID NO: 1 are modified, and having biocompatibility
(C) a peptide consisting of an amino acid sequence which has a partial sequence having 80% or more sequence identity with a partial amino acid sequence consisting of 4th to 192nd amino acid residues in the amino acid sequence set forth in SEQ ID NO: 1, and having biocompatibility,

SEQ ID NO: 1:
GAP(GAPGLQGAPGLQGMPGERGAAGLPGPKGERGDAGPKGADGAPGAP
GLQGMPGERGAAGLPGPKGERGDAGPKGADGAPGKDGVRGLAGPIGPPG
ERGAAGLPGPKGERGDAGPKGADGAPGKDGVRGLAGPIGPPGPAGAPGA
PGLQGMPGERGAAGLPGPKGERGDAGPKGADGAPGKDGVRGLAGPP)
3G.

<16>The tissue repair material kit according to any one of <1>to <15>, in which the tissue repair material is a bone tissue repair material.

<17>A tissue repair method comprising: a step of supplying the tissue repair material to a tissue-deficient part by using the tissue repair material kit according to any one of <1>to <16>.

According to the embodiment of the present disclosure, there is provided a tissue repair material kit capable of easily supplying a tissue repair material to a narrow tissue-deficient part.

According to another embodiment of the present disclosure, a tissue repair method using the tissue repair material kit is provided.

### BRIEF DESCRIPTION OF THE DRAWINGS

FIG. 1 is a schematic view showing an example of a tissue repair material kit.

FIG. 2 is a schematic view showing a syringe assembled by using the tissue repair material kit of FIG. 1.

FIG. 3 is a schematic view showing an example of the tissue repair material kit.

FIG. 4 is a schematic view showing an example of the tissue repair material kit.

### DESCRIPTION OF THE PREFERRED EMBODIMENTS

In the present disclosure, a numerical range described using “to” includes numerical values before and after “to” as a minimum value and a maximum value, respectively.

In the numerical ranges described stepwise in the present disclosure, the upper limit value or the lower limit value described in one numerical range may be replaced with the upper limit value or the lower limit value of the numerical range described stepwise in other stages. Further, in a numerical range described in the present disclosure, the upper limit value or the lower limit value of the numerical range may be replaced with the value shown in Examples.

In the present disclosure, in a case where a plurality of substances corresponding to each component in a material is present. the amount of each component in the material means the total amount of the plurality of substances present in the material, unless otherwise specified.

In the present disclosure, a combination of two or more preferred aspects is a more preferred aspect.

In the present disclosure, the term “step” includes not only an independent step but also a step that cannot be clearly distinguished from other steps, as long as the intended purpose of the step is achieved.

### Tissue Repair Material Kit

A tissue repair material kit according to the present disclosure includes a cylindrical container that has an opening portion having an opening diameter smaller than an inner diameter of the container and a tissue repair material, in which in a case where the tissue repair material is supplied, a storage elastic modulus of the tissue repair material at 25° C. is 0.1 kPa to 100 kPa.

A tissue repair material may be used for repairing a tissue-deficient part. However, a tissue-deficient part where tissue repair is required may be narrow; and there is a case where it is difficult to supply a tissue repair material to the tissue-deficient part. As described above.

for example, although JP2018-094401A discloses a bone tissue repair material as a tissue repair material, it has not considered a method of supplying the tissue repair material to a narrow tissue-deficient part.

On the other hand, the tissue repair material kit according to the present disclosure includes a cylindrical container that has an opening portion having an opening diameter smaller than an inner diameter of the container and a tissue repair material. In addition, the storage elastic modulus of the tissue repair material at 25° C. is 0.1 kPa to 100 kPa in a case of being supplied, that is, the tissue repair material is easily deformed, and thus the tissue repair material accommodated in the cylindrical container can be easily supplied to a narrow tissue-deficient part through the opening portion.

The phrase “in a case where a tissue repair material is supplied” means a case where the tissue repair material in the cylindrical container is supplied to the tissue-deficient part through the opening portion or a discharge part (described later).

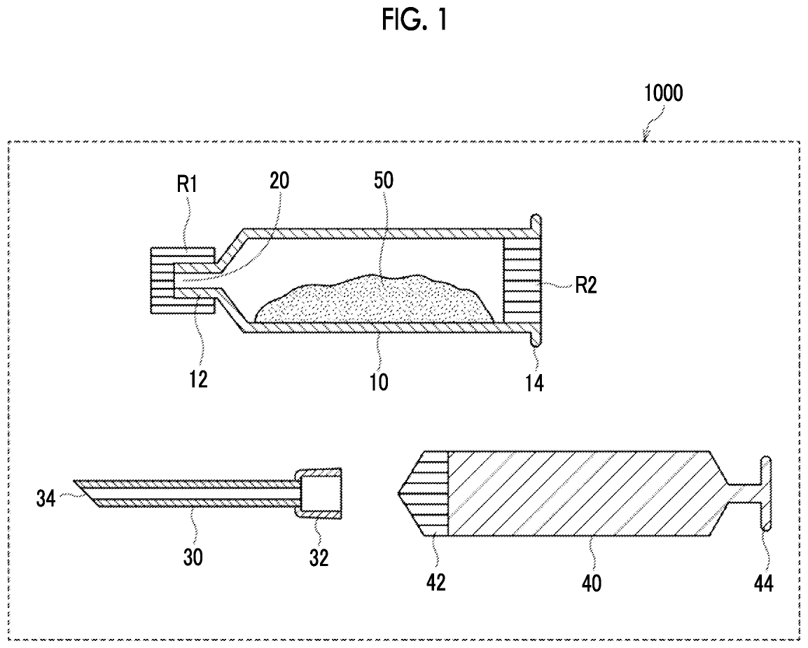

A tissue repair material kit 1000 shown in FIG. 1 as an example includes a cylindrical container 10 having an opening portion 20 having an opening diameter smaller than an inner diameter of the container, a tissue repair material 50, a discharge part 30, and a plunger 40. The opening portion 20 having one end of the cylindrical container 10 and the other end (the end part opposite to the opening portion 20) of the cylindrical container 10 are respectively sealed with rubber stoppers R1 and R2.

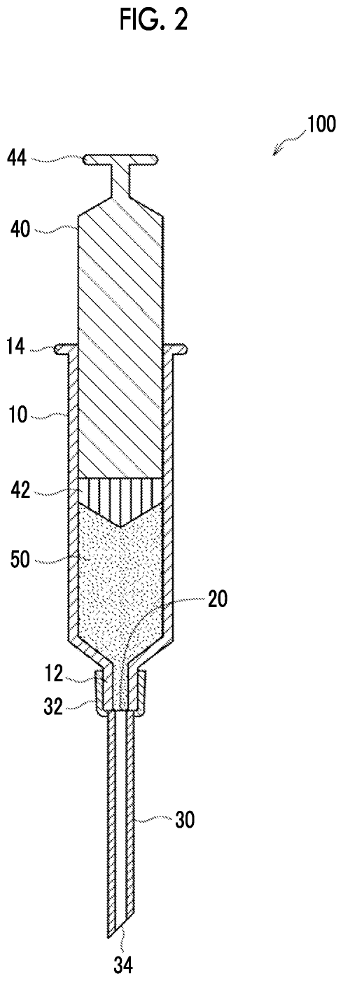

In a case of assembling the tissue repair material kit 1000, it is possible to obtain a syringe 100 shown in FIG. 2 as an example. In another aspect, the tissue repair material kit 1000 may be the syringe 100 itself assembled as described above.

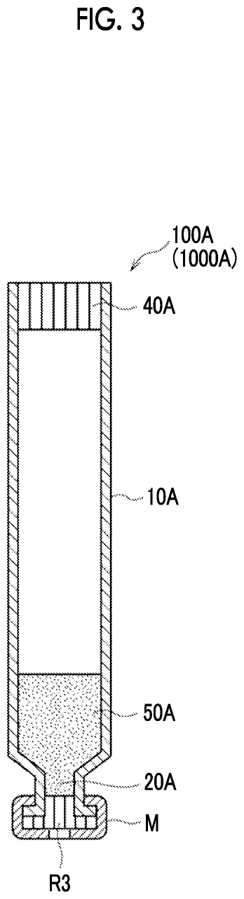

A tissue repair material kit 1000A shown in FIG. 3 as another example is a cartridge 100A that includes a cylindrical container 10A having an opening portion 20A having an opening diameter smaller than an inner diameter of the container, a tissue repair material 50A, and a plunger 40A. The opening portion 20A provided at one end (an end part opposite to the plunger 40) of the cylindrical container 10A is sealed with a rubber stopper R3. The cylindrical container 10A and the rubber stopper R3 are fixed by an aluminum member M.

The cartridge as shown in FIG. 3 can be used, for example, as a cartridge of a known syringe, similarly to a cartridge of an anesthetic which is used in a syringe for dental anesthesia.

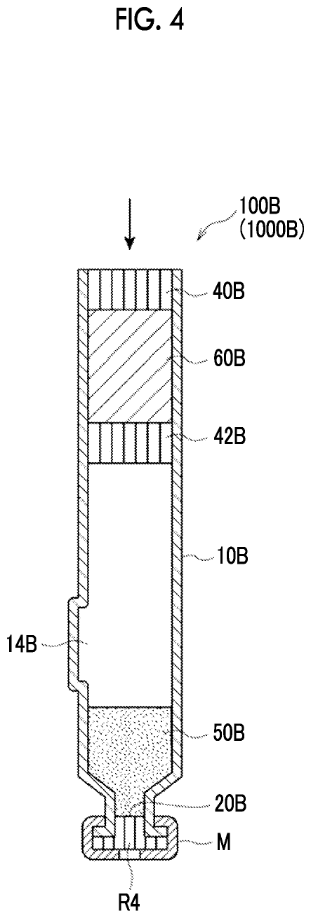

A tissue repair material kit 1000B shown in FIG. 4 as another example of the cartridge is a cartridge 100B that includes a cylindrical container 10B having an opening portion 20B having an opening diameter smaller than an inner diameter of the container, a tissue repair material 50B, plungers 40B and 42B, and a water-containing liquid 60B held between the plunger 40B and the plunger 42B. The opening portion 20B provided at one end (an end part opposite to the plunger 40B) of the cylindrical container 10B is sealed with a rubber stopper R4, and The cylindrical container 10B and the rubber stopper R4 are fixed by the aluminum member M.

A bypass 14B is provided in the cylindrical container 10B, and in a case where the plungers 40B and 42B are pressed toward the direction of the arrow and the water-containing liquid 60B reaches the bypass 14B, the water-containing liquid 60B passes through the bypass 14B and flows to the tissue repair material 50B side. This makes it possible to mix the tissue repair material 50B and the water-containing liquid 60B.

In another aspect, a filter that allows the water-containing liquid 60B to communicate with the tissue repair material 50B side may be provided in the plunger 42B, whereby the water-containing liquid 60B can be allowed to flow out to the tissue repair material 50B side through the filter of the plunger 42B without passing through the bypass 14B.

Hereinafter, each configuration will be described in more detail.

### [Cylindrical Container]

The cylindrical container has a cylindrical container having an opening portion having an opening diameter smaller than an inner diameter of the container.

The cylindrical container is a container having a cavity therein. The shape of the cylindrical container is not particularly limited. For example, it may be an elongated rod shape, and it is preferably a cylinder or a square cylinder and particularly preferably a cylinder. In addition, for example, a protruding portion including an opening portion may be integrally provided in the cylindrical container.

The cylindrical container may have a member for facilitating operation. For example, the cylindrical container has a syringe shape, and it may have, for example, a finger flange 14 as shown in FIG. 1 in order to facilitate the operation.

The material of the cylindrical container is not particularly limited, and it may be, for example, a resin, a metal, or glass. From the viewpoint of visibility, the material of the cylindrical container preferably includes a transparent resin, glass, or the like.

The inner diameter of the cylindrical container means a diameter of a circle having the same area as the area of the portion of the cavity part in the cross section perpendicular to the longitudinal direction of the cylindrical container. In a case where the inner diameter of the cylindrical container is not uniform in the longitudinal direction, the maximum value thereof is defined as the inner diameter of the cylindrical container.

The inner diameter of the cylindrical container is, for example, a value that is measured by a projection image measuring instrument, a laser displacement meter, or a caliper.

The inner diameter of the cylindrical container is, for example, preferably 5 mm to 50mm, and it is more preferably 5 mm to 30 mm from the viewpoint of handleability (ease of holding and the like).

The length of the cylindrical container (not including the opening portion) in the longitudinal direction is not particularly limited. However, it is, for example, preferably 70mm to 200 mm, and it is more preferably 70 mm to 120 mm from the viewpoint of handleability.

The opening portion is a portion that allows the tissue repair material accommodated in the cylindrical container to flow out of the cylindrical container. The opening diameter of the opening portion means a diameter of a circle having the same area as the area of the opening portion in a case of being viewed in a plan view from the outside of the cylindrical container. In a certain aspect, the opening portion may function as a portion that supplies a tissue repair material to a tissue-deficient part.

The opening diameter of the opening portion is, for example, a value that is measured by a projection image measuring instrument, a laser displacement meter, or a caliper.

From the viewpoint of reducing the force required to allow the tissue repair material to flow out of the cylindrical container, the opening diameter of the opening portion is preferably 10% to 70% and more preferably 20% to 70% or less of the inner diameter of the cylindrical container.

The opening diameter of the opening portion may be, for example, 15 mm or less. As described above, the opening portion can function as a portion that supplies a tissue repair material to a tissue-deficient part. However, in this aspect, from the viewpoint of more easily supplying the tissue repair material to a narrow tissue-deficient part, the opening diameter of the opening portion is preferably 0.8 mm to 10 mm and more preferably 0.8 mm to 6.6 mm. The upper limit of the opening diameter of the opening portion is still more preferably 5 mm or less. The opening diameter of the opening portion is even still more preferably 0.8 mm to 2.5 mm.

As will be described later, the tissue repair material may have a particulate shape in a dry state. In this aspect, From the viewpoint of reducing the force required to allow the tissue repair material to flow out of the cylindrical container, the opening diameter of the opening portion is preferably 40% to 300% of the maximum particle diameter of the tissue repair material in a dry state. As described above, the opening portion can function as a portion that supplies a tissue repair material to a tissue-deficient part. However, in this aspect, from the viewpoint of more easily supplying the tissue repair material to a narrow tissue-deficient part, the opening diameter of the opening portion is more preferably 60% to 200%. A measuring method for the maximum particle diameter will be described later.

The description that the tissue repair material is in a “dry state” is used to mean a state where the tissue repair material does not contain a liquid (for example, a water-containing liquid described below); however, moisture due to humidity in the air may be contained.

The position of the opening portion is not particularly limited and may be appropriately determined in consideration of the use application of the tissue repair material kit and the like. For example, the opening portion may be provided at one end of the cylindrical container. In this aspect, for example, a protruding portion having an inner diameter smaller than the inner diameter of the container may be integrally provided at one end of the cylindrical container, and an end part of the protruding portion may be the opening portion.

The cylindrical container may have a connecting portion (for example, a connecting portion 12 as shown in FIG. 1) for connection to a discharge part described later. In this aspect. the opening portion may be provided at an end part of the connecting portion.

The connecting portion may be integrated with the cylindrical container or may be a member that is separate from the cylindrical container.

The material of the connecting portion container is not particularly limited, and it may be, for example, a resin, a metal, or glass. From the viewpoint of visibility, the material of the cylindrical container preferably includes a transparent resin, glass, or the like.

### [Discharge Part]

The tissue repair material kit may include a discharge part that has an inner diameter smaller than the inner diameter of the cylindrical container and is provided at the opening portion. The discharge part guides the tissue repair material that has flowed out through the opening portion of the cylindrical container and allows the tissue repair material to flow out from the hole portion (for example, a hole portion 34 in FIG. 1) provided in the discharge part.

The discharge part is a member having a cavity therein and is subjected to fluid communication with the opening portion of the cylindrical container. The shape of the discharge part is not particularly limited. For example, it may be an elongated rod shape, and it is preferably a cylinder or a square cylinder and particularly preferably a cylinder. In addition, for example, the discharge part may be bent. In addition, the discharge part may have a rod shape that tapers from one end toward the other end in the longitudinal direction.

The inner diameter of the discharge part means a diameter of a circle having the same area as the area of the portion of the cavity part in the cross section perpendicular to the longitudinal direction of the discharge part. It means a diameter of a circle having the same area as the area of the hole portion through which the tissue repair material flows out from the outside of the discharge part in a case where the hole portion is viewed in a plan view: in a case where the inner diameter of the discharge part is not uniform in the longitudinal direction. For example, in a case where the discharge part (and the cavity part) has a rod shape that tapers from one end toward the other end (the hole portion from which the tissue repair material flows out) in the longitudinal direction, the inner diameter of the discharge part means the diameter of the hole portion of the tapered tip part, where the inner diameter has the minimum value.

The opening diameter of the discharge part is, for example, a value that is measured by a projection image measuring instrument, a laser displacement meter, or a caliper.

From the viewpoint of reducing the force required to allow the tissue repair material to flow out of the cylindrical container, the inner diameter of the discharge part is preferably 10% to 70% and more preferably 20% to 70% or less of the inner diameter of the cylindrical container.

In addition, since a short container which is not bulky can be obtained by making the cylindrical container thicker than the discharge part, the inner diameter of the discharge part is still more preferably 20% to 60% of the inner diameter of the cylindrical container.

The inner diameter of the discharge part may be, for example. 0.5 mm to 15 mm. From the viewpoint of more easily supplying the tissue repair material to a narrow tissue-deficient part, the inner diameter of the discharge part is preferably 0.8 mm to 10 mm and more preferably 0.8 mm to 6.6 mm. The upper limit of the inner diameter of the discharge part is still more preferably 5 mm or less. The inner diameter of the discharge part is even still more preferably 0.8 mm to 2.5 mm.

The inner diameter of the discharge part is, for example, a value that is measured by a projection image measuring instrument, a laser displacement meter, or a caliper.

As will be described later, the tissue repair material may have a particulate shape in a dry state. In this aspect, from the viewpoint of reducing the force required to allow the tissue repair material to flow out of the cylindrical container, the inner diameter of the discharge part is preferably 40% to 300% of the maximum particle diameter of the tissue repair material in a dry state. From the viewpoint of more easily supplying the tissue repair material to a narrow tissue-deficient part, the inner diameter of the discharge part is more preferably 60% to 200%. A measuring method for the maximum particle diameter will be described later.

The aspect in which the tissue repair material kit includes the discharge part is not particularly limited. The discharge part may be separated from the cylindrical container or may be connected to the cylindrical container through a connecting portion provided in the cylindrical container. For example, in the example shown in FIG. 1, the connecting portion 12 may be inserted into and connected to an insertion part 32 of the discharge part 30.

The material of the discharge part is not particularly limited: however, it may be, for example, a resin, a metal, or glass. From the viewpoint of visibility, the material of the cylindrical container preferably includes a transparent resin, glass, or the like.

### [Pressurization Unit]

The tissue repair material kit may include a pressurization unit for pushing out the tissue repair material in the cylindrical container from the opening portion or the discharge part.

For example, in an aspect in which the cylindrical container has an opening portion at one end, the tissue repair material kit may include a pressurization unit that is provided at the other end of the cylindrical container.

The pressurization unit is not particularly limited, and examples thereof include a plunger, a piston, and compressed air. Among these, from the viewpoint of facilitation, the pressurization unit is preferably a plunger.

The plunger is a member that slides on an inner wall of the cylindrical container to push out the tissue repair material accommodated in the cylindrical container to the outside of the cylindrical container. The plunger may include a gasket (for example, a gasket 42 shown in FIG. 1) for improving airtightness with respect to the inner wall of the cylindrical container and may include a pusher for entering the inside of the cylindrical container or exiting therefrom (for example, a pusher 44 shown in FIG. 1). The plunger may be a plunger that is driven by the pressure of the medium or the like. The material of the gasket is not particularly limited. For example, synthetic rubber can be used, where a lubricant may be provided. The material of the pusher is not particularly limited, and it may be, for example, a resin, a metal, or glass. In addition, the plunger may be a plunger that also serves as a gasket (for example, the plunger 40A of FIG. 3 or the plungers 40B and 42B of FIG. 4), and in this case, it is preferable to be made of rubber.

The aspect in which the tissue repair material kit includes the pressurization unit is not particularly limited. For example, the pressurization unit may be in a state of being separated from the cylindrical container or may be in a state of being provided in advance in the cylindrical container.

### [Tissue Repair Material]

The tissue repair material is a material that contributes to the formation of tissue at the implantation site by being implanted in a living body, and it may include or may not include cells. In addition, the tissue repair material may contain or may not contain a component which promotes a reaction in a living body, such as a growth factor, a drug, or the like. In addition, a tissue repair material, which has been mixed or subjected to form a composite with an inorganic material such as hydroxyapatite, may be applied. Further, the tissue repair material is not necessarily a tissue repair material that contributes to the formation of the normal tissue which is normally present in a tissue-deficient part (for example, an implantation site) and a tissue repair material that also contains a material which promotes the formation of the abnormal tissue including the scar tissue and the like.

The tissue repair material may be, for example, a bone tissue repair material. The bone tissue repair material is a material that is supplied to a bone-deficient part of a living body or a site in which bone morphogenesis is desired, and due to having a certain biocompatibility and biodegradability, the strength thereof is ensured for a desired period of time. It contributes to the formation of the bone tissue while maintaining the volume of the supply site, thereby being capable of being a place of replacement with the formed bone tissue. As a result, it is presumed that the bone tissue repair material is arranged, whereby the formation of the tissue such as the bone proceeds favorably. However, the present disclosure is not restricted by the theory of this presumption. It is preferable that the bone tissue repair material has sufficient biodegradability and bone replaceability to replace the bone tissue.

The shape of the tissue repair material is not particularly limited, and it may be, for example, a particulate shape or a block shape. The particulate shape means a shape of a granule, a powder, a slurry; or the like.

The shape of the granule is not particularly limited, and it may be, for example, an irregular shape, a spherical shape, a powdery shape, a porous shape, a fibrous shape, a spindle shape, a flat shape, a sheet shape, or the like. The shape of the granule is preferably an irregular shape, a spherical shape, a powdery shape, or a porous shape. The “irregular shape” means a shape having a non-uniform surface, and examples thereof include a shape having unevenness, such as a shape of rock.

In a case where the tissue repair material has a particulate shape, the particle diameter of the tissue repair material means the size of each particle. The maximum particle diameter is a maximum value of the sizes of a plurality of particles. The size of a grain is defined as a square root of a projected area, that is, a length of one side of a square having the same area as the projected area of the particle. The projected area is calculated as an area of a region corresponding to the tissue repair material by carrying out image processing to binarize an image captured by placing a grain on a background of a brightness different from that of the particle. Examples of the analysis apparatus that can be used for imaging and image processing include “Morphologi 3G” (Spectris plc).

The aspect in which the tissue repair material kit includes the tissue repair material is not particularly limited. For example, the tissue repair material may be provided in the tissue repair material kit in a state of being accommodated in a packaging material or the like, and the tissue repair material may be accommodated in the cylindrical container in a case of using the tissue repair material kit.

In another aspect, the tissue repair material may be accommodated in the cylindrical container in advance, which is preferable from the viewpoint of workability.

From the viewpoint of improving the fluidity of the tissue repair material, the water absorption rate of the tissue repair material is preferably 300% to 2,000%, more preferably 400% to 2,000%, and particularly, still more preferably 500% to 2,000%.

It is preferable that the tissue repair material has a water absorption rate equal to or higher than a certain level. For example, in a case of a tissue repair material containing gelatin granules (for example, a bone tissue repair material), it is preferable to exhibit a water absorption rate of 300% or more. In a case where the water absorption rate is set to 300% or more, it is easy to obtain favorable tissue repair ability. From the viewpoint of holding blood clotting during tissue repair, the water absorption rate of the tissue repair material is preferably 400% or more and more preferably 500% or more. The upper limit of the water absorption rate of the tissue repair material is not particularly limited: however, it is preferably 2,000% or less. In a case where the tissue repair material contains only gelatin granules, the water absorption rate of the tissue repair material is defined as the water absorption rate of the gelatin granules.

The water absorption rate of the tissue repair material means physical properties that are measured as follows. In each of three filter cups of which the tare mass has been measured in advance (each having a volume of 500 μL, which includes a filter having a pore diameter of 0.22 μm on the bottom surface: hereinafter, referred to as a container), 10.0 mg of each test substance (tissue repair material) is collected (n=3). A sufficient amount of water is added thereto, followed by mixing until the water absorption by the test substance is saturated (rotation: 2 hours, at an environmental temperature). Next, centrifugation (6,000× g. 1 minute, 25° C.) is carried out to remove excess water, and the mass of the container containing the test substance after water absorption (the total mass after water absorption) is measured. Separately, a blank test is carried out three times in the same manner as described above except that the test substance is not used, and a value obtained by subtracting the tare mass of the container from the mass of the container, to which water has adhered, is defined as the residual water amount. The average value from the three times of measurements of the residual water amount is defined as the residual water amount of the blank test, and a value obtained by subtracting the residual water amount and the tare mass of the container from the total mass after the water absorption is defined as the mass of the test substance after water absorption. The mass of the test substance after water absorption is divided by the mass of the test substance before water absorption to define the water absorption rate (%).

The tissue repair material kit may include a water-containing liquid that is to be mixed with the tissue repair material. The water-containing liquid may be, for example, a solution or a dispersion liquid, which uses water as a solvent.

The water-containing liquid is not particularly limited, and it may be, for example, water for injection, saline, a hyaluronic acid aqueous solution, a carboxymethyl cellulose aqueous solution, a polyethylene glycol aqueous solution, a growth factor aqueous solution, an antibiotic aqueous solution, blood, a bone marrow fluid, a platelet-rich blood plasma, or a mixture thereof.

The aspect in which the tissue repair material kit includes the water-containing liquid is not particularly limited. For example, the water-containing liquid may be provided in the tissue repair material kit in a state of being accommodated in a packaging material or the like, and the water-containing liquid may be mixed with the tissue repair material in a case of using the tissue repair material kit.

In another aspect, the water-containing liquid may be mixed with the tissue repair material in advance.

The content of the water-containing liquid is preferably 50% by mass to 2,000% by mass and more preferably 100% by mass to 2,000% by mass with respect to the dry mass of the tissue repair material. A state where the water-containing liquid is mixed with the tissue repair material and thus the mass of the tissue repair material is increased may be referred to as a hydrous state.

In a case where a tissue repair material is supplied, (that is, a case where the tissue repair material in the cylindrical container is supplied to the tissue-deficient part through the opening portion or a discharge part), the storage elastic modulus of the tissue repair material at 25° C. is 100 kPa or less, and it is more preferably 50 kPa or less. This makes the tissue repair material easily deformed in a case where the tissue repair material is supplied, which prevents the tissue repair material from being bridged and clogged at the opening portion or an upper part of the discharge part or prevents the tissue repair materials from being pushed with each other to be crushed and destroyed.

The lower limit of the storage elastic modulus of the tissue repair material at 25° C. is not particularly limited from the viewpoint of the above-described effect: however, it is set to 0.1 kPa or more from the viewpoint of handleability.

In addition, from the viewpoint of easily maintaining the shape of the tissue repair material supplied to the tissue-deficient part, the storage elastic modulus is preferably 0.1 kPa to 50 kPa.

The storage elastic modulus is measured as follows. A test substance is placed in a SUS ring having an inner diameter of 15 mm and a height of 7.5 mm, a vibratory flow is applied thereto in a shear direction while carrying out pressing with a constant load of 1 N by using a disposable parallel plate (Φ12 mm) of a rheometer (Anton Paar, MCR302), and a linear viscoelasticity measurement is carried out. The amount of the test substance is set such that the height thereof in a case of being pressed is 3.5 mm or more and 4.5 mm or less. The angular frequency is swept from 1 rad/sec to 50 rad/sec and then swept from 50 rad/sec to 1 rad/sec, and a storage elastic modulus at 25° C. at 1 rad/sec in the latter sweep is measured (n=2 to 3).

Under the following conditions, the storage elastic modulus of the tissue repair material at 25° C. is preferably 100 kPa or less and more preferably 50 kPa or less. This makes it easy to reduce the force required to allow the tissue repair material to flow out of the cylindrical container. Under the following conditions, the lower limit of the storage elastic modulus of the tissue repair material at 25° C. is not particularly limited from the viewpoint of the above-described effect: however, it is preferably set to 0.1 kPa or more from the viewpoint of handleability:

### [Conditions]

In a case where the tissue repair material contains the water-containing liquid, a value obtained by multiplying a mass fraction of a content of the water-containing liquid with respect to a dry mass of the tissue repair material by a tap density of the tissue repair material in a dry state is 0.40 g/mL to 0.70 g/mL.

The tap density is measured as follows. A test substance is placed up to 5 mL in a 10mL graduated cylinder of which the tare mass has been measured in advance, the mass of the graduated cylinder containing the test substance is weighed, and a value obtained by subtracting the tare mass is defined as the mass of the test substance. This graduated cylinder is tapped 50times or more until the volume of the test substance becomes constant, and the scale of the graduated cylinder is read to measure the volume after the tapping. The mass of the test substance is divided by the volume after tapping to obtain the tap density (g/mL).

The tissue repair material may contain a biocompatible polymer.

The “polymer” is a molecule having a large molecular weight and refers to a molecule having a structure including a large number of repetitions of a unit that is obtained substantially or conceptually from a molecule having a small molecular weight. The “polymer” is preferably a compound having a weight-average molecular weight of 10,000 or more. Examples of the polymer include a polyamine, a polysaccharide, a polypeptide, a protein, a polyamide, a polyester, a polyolefin, a polyether, and a polynucleotide.

The “biocompatibility” means a property that does not cause a significantly harmful response such as a long-term and chronic inflammatory reaction, during contact with a living body: Examples of the substance having biocompatibility include a protein and polysaccharides.

From the viewpoint of being used as a tissue repair material, the tissue repair material preferably contains a biodegradable polymer. Examples of the biodegradable polymer include polypeptides such as a naturally derived peptide, a recombinant peptide, and a chemically synthesized peptide (for example, gelatin described later). In addition, examples thereof include polylactic acid, polyglycolic acid, a lactic acid/glycolic acid copolymer (PLGA), hyaluronic acid, glycosaminoglycan, proteoglycan, chondroitin, cellulose, agarose, carboxymethylcellulose, chitin, and chitosan. Among the above, It is particularly preferable that the tissue repair material in the present embodiment contains a recombinant peptide. Examples of the non-biodegradable polymer include polytetrafluoroethylene (PTFE), polyurethane, polypropylene, polyester, vinyl chloride, polycarbonate, acryl, silicone, and 2-methacryloyloxyethyl phosphoryl choline (MPC).

The tissue repair material is preferably manufactured from a biocompatible polymer, more preferably manufactured from recombinant gelatin, and particularly preferably manufactured from CBE3 (set forth in SEQ ID NO: 1).

The kind of the polypeptide such as a recombinant peptide or a chemically synthesized peptide is not particularly limited as long as the polypeptide has biocompatibility and biodegradability. Examples of such a polypeptide include gelatin, collagen, elastin, fibronectin, ProNectin, laminin, tenascin, fibrin, fibroin, entactin, thrombospondin, and RetroNectin. Among these, the tissue repair material in the present embodiment preferably contains gelatin, collagen, or atelocollagen, more preferably contains natural gelatin, recombinant gelatin, or chemically synthesized gelatin, and still more preferably contains recombinant gelatin. The natural gelatin referred to herein means gelatin produced from naturally derived collagen.

Examples of the natural gelatin and the recombinant gelatin thereof include gelatins derived from animals such as a fish and a mammal, where gelatin derived from a mammalian animal is preferable. Examples of the mammalian animal include a human, a horse, a pig, a mouse, and a rat. In a case where the tissue repair material according to the present embodiment contains recombinant gelatin, it is more preferable that the recombinant gelatin is derived from a human.

Hereinafter, an amino acid sequence constituting a polypeptide is expressed by using one-letter notation (for example, “G” in a case of a glycine residue) or three-letter notation (for example. “Gly” in a case of a glycine residue), which is well known in the art. In addition. unless otherwise specified. “%” regarding an amino acid sequence of a polypeptide is based on the number of amino acid (or imino acid) residues.

The recombinant gelatin means a polypeptide or a protein-like substance which has an amino acid sequence similar to that of gelatin produced according to a gene recombination technique. The following recombinant gelatin is implanted in a living body, whereby it can be used as a tissue repair material which contributes to the formation of tissue at the implanted site. The “tissue” that can be repaired by the tissue repair material may be hard tissue such as teeth and bone, and the following recombinant gelatin is particularly suitable as a bone tissue repair material.

The recombinant gelatin preferably has a repetition of a sequence represented by Gly-X-Y which is characteristic of collagen. Here, a plurality of pieces of Gly-X-Y may be the same or different from each other. In Gly-X-Y, Gly represents a glycine residue, and X and Y represent any amino acid residue other than the glycine residue. It is preferable that X and Y include a large amount of imino acid residues, that is, proline residues or oxyproline residues. The content of such imino acid residues preferably occupies 10% to 45% of the entire gelatin. The content of Gly-X-Y in the gelatin is preferably 80% or more, more preferably 95% or more, and still more preferably 99% or more with respect to the entire gelatin.

For example, recombinant gelatin disclosed in EP1014176A, U.S. Pat. No. 6,992,172B, WO2004/85473A, WO2008/103041A, JP2010-519293A, JP2010-519252A, JP2010-518833A, JP2010-519251A, WO2010/128672A, WO2010/147109A, and the like can be used as the recombinant gelatin, the recombinant gelatin is not limited thereto.

The molecular weight of the recombinant gelatin is preferably 2 kDa to 100 kDa, more preferably 5 kDa to 90 kDa or less, and still more preferably has a molecular weight of 10 kDa to 90 kDa.

In addition, from the viewpoint of biocompatibility, the recombinant gelatin is preferably recombinant gelatin that further contains a cell adhesion signal and the recombinant gelatin is more preferably recombinant gelatin that has, in one molecule, two or more cell adhesion signals which are present in the recombinant gelatin. Examples of such a cell adhesion signal include sequences such as an RGD sequence, a YIGSR (SEQ ID NO: 2) sequence, a PDSGR (SEQ ID NO: 3) sequence, an LGTIPG (SEQ ID NO: 4) sequence, an IKVAV (SEQ ID NO: 5) sequence, and an HAV sequence. Among these, an RGD sequence is preferable, and an ERGD (SEQ ID NO: 6) sequence among the RGD sequences is more preferable.

However, it is preferable that the sequence of the recombinant gelatin satisfies at least one of the following aspects (1-1) to (1-3). It is noted that the recombinant gelatin may singly have the following aspects (1-1) to (1-3) or may have a combination of two or more of the aspects.
(1-1) It does not include a serine residue and a threonine residue.
(1-2) It does not include a serine residue, a threonine residue, an asparagine residue, a tyrosine residue, and a cysteine residue.
(1-3) It does not include an amino acid sequence represented by Asp-Arg-Gly-Asp (SEQ ID NO: 7).

It is preferable that the recombinant gelatin has a repeating structure of A-[(Gly-X-Y)ₙ]ₘ-B (SEQ ID NO: 8). m represents an integer of 2 to 10, and it is preferably 3 to 5. A and B represent any amino acid or amino acid sequence. n represents an integer of 3 to 100,and it is preferably 15 to 70 and more preferably 50 to 60.

It is more preferable that the recombinant gelatin is represented by a formula: Gly-Ala-Pro-[(Gly-X-Y)₆₃]₃-Gly (SEQ ID NO: 9). In the formula, 63 pieces of X's each independently represent any amino acid residue, and 63 pieces of Y's each independently represent any amino acid residue. It is noted that 3 pieces of [(Gly-X-Y)₆₃](SEQ ID NO: 10) may be the same or different from each other.

It is preferable that the recombinant gelatin satisfies at least one of the following aspects (2-1) to (2-4). It is noted that the recombinant gelatin may singly have the following aspects (2-1) to (2-4) or may have a combination of two or more of the aspects.
(2-1) It is such that the carbamoyl group has not been hydrolyzed.
(2-2) It does not include procollagen.
(2-3) It does not include telopeptide.
(2-4) It is a substantially pure collagen-like material prepared from a nucleic acid encoding natural collagen.

From the viewpoint of high tissue repair ability, the recombinant peptide preferably includes the following peptide (A), (B), or (C).
(A) A peptide consisting of an amino acid sequence set forth in SEQ ID NO: 1.
(B) A peptide consisting of an amino acid sequence in which one or several amino acid residues in the amino acid sequence set forth in SEQ ID NO: 1 are modified (for example, deleted, substituted, or added), and having biocompatibility.
(C) A peptide consisting of an amino acid sequence which has a partial sequence having 80% or more sequence identity with a partial amino acid sequence consisting of 4th to 192nd amino acid residues in the amino acid sequence set forth in SEQ ID NO: 1, and having biocompatibility.

SEQ ID NO: 1:
GAP(GAPGLQGAPGLQGMPGERGAAGLPGPKGERGDAGPKGADGAPGAP
GLQGMPGERGAAGLPGPKGERGDAGPKGADGAPGKDGVRGLAGPIGPPG
ERGAAGLPGPKGERGDAGPKGADGAPGKDGVRGLAGPIGPPGPAGAPGA
PGLQGMPGERGAAGLPGPKGERGDAGPKGADGAPGKDGVRGLAGPP)3G

In the peptide defined in (B) described above, the number of amino acid residues to be modified (for example, deleted, substituted, or added) varies depending on the total number of amino acid residues in the recombinant gelatin: however, it is preferably 2 to 15 and more preferably 2 to 5.

In (C) described above, the “sequence identity” regarding the amino acid sequences of the two kinds of peptides to be compared (that is, the peptide of (A) and the peptide of (C)) refers to a value calculated according to the following expression. It is noted that the comparison (alignment) of a plurality of peptides is carried out according to a conventional method so that the number of identical amino acid residues is the largest.

Sequence identity (%)=[(number of identical amino acid residues)/(alignment length)]×100

In (C) described above, the “partial amino acid sequence consisting of 4th to 192nd amino acid residues” corresponds to a repeating unit in the amino acid sequence set forth in SEQ ID NO: 1. The “partial sequence” corresponds to a repeating unit in the sequence of (C) described above. It is sufficient that the peptide of (C) described above includes at least one repeating unit (partial sequence) having 80% or more sequence identity with the repeating unit in the amino acid sequence set forth in SEQ ID NO: 1. and it is preferable to include two or more thereof.

It is noted that in a case where the peptide of (C) described above includes a plurality of different repeating units, some of the plurality of repeating units may be such that the sequence identity with the repeating unit in the amino acid sequence set forth in SEQ ID NO: 1 is less than 80%. However, in the peptide of (C) described above, it is preferable that the total number of amino acid residues of the repeating unit (partial sequence) having 80% or more sequence identity with the repeating unit in the amino acid sequence set forth in SEQ ID NO: 1 is 80% or more of the total number of amino acid residues.

In addition, from the viewpoint of tissue repair ability, the sequence identity of the partial sequence in the peptide of (C) with the partial amino acid sequence consisting of 4th to 192nd amino acid residues in the amino acid sequence set forth in SEQ ID NO: 1 is preferably 90% or more and more preferably 95% or more.

The length of the peptide defined in (C) described above is such that the number of amino acid residues can be set to 151 to 2,260, where it is preferably 193 or more from the viewpoint of the decomposability after crosslinking, and it is preferably 944 or less from the viewpoint of stability. In addition, the number of amino acid residues is more preferably 380 to 756.

The recombinant gelatin described above can be produced by a gene recombination technique known to those skilled in the art, and it may be produced, for example, according to the method described in EP1014176A, U.S. Pat. No. 6,992,172B, WO2004/85473A, WO2008/103041A, or the like. Specifically, a gene encoding an amino acid sequence of a predetermined recombinant gelatin is obtained, the gene is incorporated into an expression vector to prepare a recombinant expression vector, and the expression vector is introduced into a proper host to prepare a transformant. The recombinant gelatin is produced by culturing the obtained transformant in an appropriate medium. Therefore, it is possible to prepare the recombinant gelatin that is used in the present disclosure by collecting the recombinant gelatin produced from a culture product.

The tissue repair material is not limited to a tissue repair material consisting of one material such as recombinant gelatin. For example, in addition to the recombinant gelatin, components which promote reactions in a living body, such as a growth factor and a drug, and other components such as cells, which can contribute to the repair or regeneration of the tissue, may be contained. Examples of such a component include a component associated with bone regeneration or osteogenesis, such as a bone-inducing agent. Examples of the bone-inducing agent include bone morphogenic factor (bone morphogenic protein: BMP) and basic fibroblast growth factor (bFGF), which are not particularly limited. In addition, a tissue repair material, which has been mixed or subjected to form a composite with an inorganic material such as hydroxyapatite, may be applied.

A manufacturing method for the tissue repair material kit is not particularly limited. For example, the tissue repair material kit may be manufactured by preparing the cylindrical container, the tissue repair material, and the like by methods that are generally used in the present technical field, and then packaging these, for example, in one package.

### Tissue Repair Method

A tissue repair method according to the present disclosure includes a step of supplying the tissue repair material to a tissue-deficient part by using the tissue repair material kit according to the present disclosure.

The step of supplying the tissue repair material may be appropriately carried out in consideration of the configuration of the tissue repair material kit and the like.

For example, the tissue repair material kit 1000 shown in FIG. 1 is used to assemble the syringe 100 shown in FIG. 4. The hole portion 34 of the discharge part 30 is inserted in the vicinity of the tissue-deficient part. Then, the plunger 40 is pressed into the cylindrical container 10 to supply the tissue repair material 50 to the tissue-deficient part through the hole portion 34.

In this way, it is possible to supply the tissue repair material to a narrow tissue-deficient part.

### EXAMPLES

Hereinafter, the present disclosure will be described in more detail with reference to Examples. However, the present disclosure is not limited to these Examples.

### Example 1

### (Recombinant Gelatin)

In the present Example, a recombinant peptide CBE3 was used as a biocompatible polymer contained in a tissue repair material. As the CBE3, the following one described in WO2008/103041A was used.
Molecular weight: 51.6 kD
Structure: GAP[(GXY)₆₃]₃G (SEQ ID NO: 9)
Number of amino acids: 571 amino acids
RGD sequence: 12 sequences
Imino acid content: 33%
Almost 100% of amino acids have a repeating structure of GXY.
CBE3 has an ERGD (SEQ ID NO: 6) sequence.

The amino acid sequence of the CBE3 does not include a serine residue, a threonine residue, an asparagine residue, a tyrosine residue, and a cysteine residue.
Isoelectric point: 9.34
Hydrophilic repeating unit ratio in polymer: 26.1%

Amino acid sequence
(SEQ ID NO: 1)
GAP(GAPGLQGAPGLQGMPGERGAAGLPGPKGERGDAGPKGADGAPGAP
GLQGMPGERGAAGLPGPKGERGDAGPKGADGAPGKDGVRGLAGPIGPPG
ERGAAGLPGPKGERGDAGPKGADGAPGKDGVRGLAGPIGPPGPAGAPGA
PGLQGMPGERGAAGLPGPKGERGDAGPKGADGAPGKDGVRGLAGPP)3G

### (Container Used to Freeze Recombinant Gelatin Solution)

A cylindrical cup-shaped container made of an aluminum alloy (JIS A5056 alloy) was prepared. The container had a shape in which a side surface and a bottom surface were closed and a top surface was open in a case where a curved surface was regarded as the side surface. The bottom surface had a thickness of 5 mm, and an inner circumference of the bottom surface was chamfered with R2 mm. The insides of the side surface and the bottom surface were coated with a tetrafluoroethylene/hexafluoropropylene copolymer (FEP), and as a result, the inner diameter of the container was 104 mm. Hereinafter, this container will be referred to as a “cylindrical container”.

### (Preparation of Tissue Repair Material)

A solution containing the above-described recombinant gelatin was purified and then concentrated to 4.0% by mass by ultrafiltration at 30° C. The obtained gelatin aqueous solution was lyophilized to obtain a lyophilized body 1. Water for injection was added to the lyophilized body 1, the temperature was raised to 37° C. over 30 minutes, and the resultant was redissolved to obtain again a 7.5% by mass gelatin aqueous solution. Approximately 20 g of the gelatin aqueous solution was poured into cylindrical containers, and then 14 pieces of the cylindrical containers were placed, through a 1 mm-thick glass plate, on a 350×634×20 mm aluminum plate precooled to approximately −35° C., covered with a lid, and allowed to stand for 1 hour to obtain a frozen body of the gelatin. The frozen body of the gelatin was lyophilized using a lyophilizer (DFR-5N-B manufactured by ULVAC, Inc.) to remove water, thereby preparing a lyophilized body 2.

The lyophilized body 2 was manually divided into a size of 20 mm square or less and pulverized using a screen mill (Comil U10, manufactured by Quadro Engineering). The pulverization was carried out twice using screens having pore diameters different from each other. In the first pulverization, a screen having a pore diameter of 0.079 inches was used as the screen, and a rotary impeller was rotated at a rotation speed of 1,100±10 rpm to carry out pulverization. As the rotary impeller, a rake type (7A1611) was used. In the second pulverization, a screen having a pore diameter of 0.040 inches was used as the screen. The rotary impeller was rotated at a rotation speed of 2,200±10 rpm to carry out pulverization.

After the second pulverization, granules on a sieve, which had been obtained after sieving at a sieve opening of 1.4 mm and a sieve opening of 0.3 mm, were collected, and 89.5 mg thereof was used to fill a glass vial (barrel diameter: 24.3 mm) of 10 mL was by using a filling machine (Aishin Nano Technologies Co., Ltd., TF-70AD). The same vial was installed in a clean oven (Nitto Rika Kogyo Co., Ltd., NCO-500A600L-WS) and subjected to a heating treatment in a nitrogen atmosphere at 135° C. for 5 hours to obtain a specimen I as a granular tissue repair material. Table 1 shows the results of measuring the particle diameter, the water absorption rate, the storage elastic modulus at 25° C. (hereinafter, may be simply referred to as a “storage elastic modulus”), and the tap density. For the particle diameter, 89.5 mg of the specimen 1 was subjected to measurement in a dry state using an image examination device (Morphologi G3, Spectris plc). The storage elastic modulus was measured in a state where an amount of 333% by mass of the water for injection was added to the specimen 1 and a state where an amount of 222% by mass of the water for injection was added to the specimen 1.

The storage elastic modulus was measured as follows. A test substance was placed in a SUS ring having an inner diameter of 15 mm and a height of 7.5 mm, a vibratory flow was applied thereto in a shear direction while carrying out pressing with a constant load of 1 N by using a disposable parallel plate (Φ12 mm) of a rheometer (Anton Paar, MCR302), and a linear viscoelasticity measurement was carried out. The amount of the test substance was set such that the height thereof in a case of being pressed was 3.5 mm or more and 4.5 mm or less. The angular frequency was swept from I rad/sec to 50 rad/sec and then swept from 50 rad/sec to 1rad/sec, and a storage elastic modulus at 1 rad/sec in the latter sweep was measured. The above measurement was repeated three times, and the average value therefrom was defined as the storage elastic modulus of the test substance.

The tap density was measured as follows. A test substance was placed up to 5 mL in a 10 mL graduated cylinder of which the tare mass had been measured in advance, the mass of the graduated cylinder containing the test substance was weighed, and a value obtained by subtracting the tare mass was defined as the mass of the test substance. This graduated cylinder was tapped 50 times or more until the volume of the test substance became constant, and the scale of the graduated cylinder was read to measure the volume after the tapping. The mass of the test substance was divided by the volume after tapping to obtain the tap density (g/mL).

### (Discharge Test)

A syringe “SS-01T” (a syringe main body including a cylindrical container having an opening portion that has an opening diameter smaller than the inner diameter of the container and a plunger, inner diameter of container: 6.6 mm, opening diameter of opening portion of protruding portion: 1.2 mm, total length: 94.6 mm) manufactured by TERUMO CORPORATION was used to prepare tissue repair material kits 1 to 6.
(1) Tissue repair material kit 1
   Syringe “SS-01T” (cylindrical container, plunger)
   Specimen 1 (tissue repair material)
   24G needle (discharge part, inner diameter of discharge part: 0.3 mm)
(2) Tissue repair material kit 2
   Syringe “SS-01T” (cylindrical container, plunger)
   Specimen 1 (tissue repair material)
   20G needle (discharge part, inner diameter of discharge part: 0.6 mm)
(3) Tissue repair material kit 3
   Syringe “SS-01T” (cylindrical container, plunger)
   Specimen 1 (tissue repair material)
   18G needle (discharge part, inner diameter of discharge part: 0.8 mm)
(4) Tissue repair material kit 4
   Syringe “SS-01T” (cylindrical container, plunger)
   Specimen 1 (tissue repair material)
(5) Tissue repair material kit 5
   Syringe “SS-01T” (cylindrical container, plunger) in which the opening diameter of the opening portion has been expanded to 2.5 mm with a drill.
   Specimen 1 (tissue repair material)
(6) Tissue repair material kit 6
   Syringe “SS-01T” (cylindrical container, plunger) in which the opening diameter of the opening portion has been expanded to 4.5 mm with a drill.
   Specimen 1 (tissue repair material)

In addition, the following tissue repair material kit 7 was prepared for the reference experiment.
(7) Tissue repair material kit 7 for reference experiment
   Syringe “SS-01T” (cylindrical container, plunger) in which the opening diameter of the opening portion has been expanded to 6.6 mm with a drill.
   Specimen 1 (tissue repair material)

A syringe was filled with 80.0 mg of the specimen 1, and an amount of 333% by mass of the water for injection was added to the specimen 1 to swell the specimen 1. The protruding portion is integrally connected to the cylindrical container and includes the opening portion. However, it functions as a connecting portion in a case where a needle (discharge part) is attached as in the tissue repair material kits 1 to 3. The tissue repair material kits 4 to 7 are examples in which the needle (discharge part) is not provided.

A piston of each syringe was pushed with an electric measurement stand (MX-1000N-FA-CN, manufactured by IMADA Co., Ltd.) to which a force gauge (ZP-200N, manufactured by IMADA Co., Ltd.) was attached, and the dischargeability was evaluated, and the maximum load required for discharge was measured. Table 2 shows the results of repeating the operation three times with each syringe. Those which have not been subjected to the measurement are indicated as “-”.

Table 3 shows the ratio of the opening diameter of the opening portion or the inner diameter of the discharge part to the maximum particle diameter of the specimen 1 in the dry state.

In addition, Table 4 shows the ratio of the opening diameter of the opening portion or the inner diameter of the discharge part to the inner diameter of the cylindrical container.

### Comparative Example 1

As a bone tissue repair material, B TCP granules (CURASAN, CERASORB M) were prepared. Table 1 shows the results of measuring the particle diameter, the water absorption rate, the storage elastic modulus, and the tap density in the same manner as in Example 1. The storage elastic modulus was measured in a state where an amount of 77% by mass of the water for injection was added to BTCP.

The reason why the amount of the tissue repair material and the amount of the water are different between Example 1 and Comparative Example 1 is to equalize the amount of water per unit volume of the tissue repair material in the hydrous state.

### (Discharge Test)

Table 2 shows the results of carrying out a discharge test in the same manner as in Example 1. In addition, Table 3 shows the ratios of the inner diameter of the discharge part and the opening diameter of the opening portion to the maximum particle diameter of the BTCP granules in the dry state.

### Comparative Example 2

A specimen 1 was prepared as a bone tissue repair material. Table 1 shows the results of measuring the storage elastic modulus in the same manner as in Example 1 except that the water for injection was not added to the specimen 1 (the particle diameter and the water absorption rate are the same values as those in Example 1)

### (Discharge Test)

Table 2 shows the results of carrying out a discharge test in the same manner as in Example 1 except that the water for injection was not added to the specimen 1. It is noted that the ratios of the inner diameter of the discharge part and the opening diameter of the opening portion to the maximum particle diameter in the dry state are the same as those in Example 1, and thus the description thereof is omitted in Table 3.

TABLE 1

| | Particle diameter in dry state (μm) | | | | Water | |
|---|---|---|---|---|---|---|
| | Minimum | Medium | | Storage elastic | absorption | Tap |
| | value | value | Maximum | modulus | rate | density |
| | (reference) | (reference) | value | (kPa) | (%) | (g/mL) |
| Example 1: specimen 1 | 13.2 | 1220 | 1900.5 | 46 (333% by mass, | 558 | 0.199 |
| (with water addition) | | | | water added) | | |
| | | | | 41 (222% by mass, | | |
| | | | | water added) | | |
| Comparative Example 1: | 13.0 | 957.4 | 1269.5 | 1999 (77% by | 99 | 0.859 |
| βTCP granule | | | | mass, water added) | | |
| Comparative Example 2: | 13.2 | 1220 | 1900.5 | 529 (without water | 558 | 0.199 |
| specimen 1 (without water | | | | addition) | | |
| addition) | | | | | | |

TABLE 2

| Opening diameter of opening | Force required for discharge (N) | | | | |
|---|---|---|---|---|---|
| portion or inner diameter of | | | | | 6.6 mm |
| discharge part | 0.8 mm | 1.2 mm | 2.5 mm | 4.0 mm | (reference) |
| Example 1: specimen 1 (with | 57.9 | 12.8 | 5.4 | 4.1 | 1.8 |
| water addition) | | | | | |
| Comparative Example 1: βTCP | Cannot be | Cannot be | Cannot be | Cannot be | 3.0 |
| granule | discharged | discharged | discharged | discharged | |
| Comparative Example 2: | Cannot be | Cannot be | Cannot be | Cannot be | 3.2 |
| specimen 1 (without water | discharged | discharged | discharged | discharged | |
| addition) | | | | | |

TABLE 3

| Opening diameter of opening | Ratio of inner diameter of discharge part and opening diameter of opening | | | | | |
|---|---|---|---|---|---|---|
| portion or inner diameter of | portion with respect to maximum particle diameter in dry state | | | | | |
| discharge part | 0.3 mm | 0.6 mm | 0.8 mm | 1.2 mm | 2.5 mm | 4.0 mm |
| Example 1: specimen 1 (with | 16% | 32% | 42% | 63% | 132% | 210% |
| water addition) | | | | | | |
| Comparative Example 1: | 24% | 47% | 63% | 95% | 197% | 315% |
| βTCP granule | | | | | | |

TABLE 4

| Opening diameter of opening | Ratio of opening diameter of opening portion or inner diameter of discharge | | | | | |
|---|---|---|---|---|---|---|
| portion or inner diameter of | part with respect to inner diameter of cylindrical container | | | | | |
| discharge part | 0.3 mm | 0.6 mm | 0.8 mm | 1.2 mm | 2.5 mm | 4.0 mm |
| Example 1: specimen 1 (with | 5% | 9% | 12% | 18% | 38% | 61% |
| water addition) | | | | | | |

In Example 1, the storage elastic modulus of the tissue repair material in a case where the tissue repair material is supplied is 41 kPa to 46 kPa, which is in the range specified by the present application, the tissue repair material is capable of being discharged at all of the opening diameters of the opening portions and the inner diameters of the discharge parts, and thus it is possible to easily supply the tissue repair material to a narrow tissue-deficient part.

In addition, as can be seen from Table 2, the pressure required for discharge is as small as 57.9 N or less in Example 1, and thus the tissue repair material can be supplied with a smaller force.

In addition, as can be seen from Table 4, in a case where the opening diameter of the opening portion and the inner diameter of the discharge part are 0.8 mm to 4.0 mm, the ratio of the diameter to the inner diameter of the cylindrical container is 12% to 61%, which is within a preferred range. From the results shown in Table 2, it can be seen that the tissue repair material can be supplied with a smaller force.

On the other hand, in Comparative Example 1 and Comparative Example 2, the storage elastic moduli of the tissue repair material in a case where the tissue repair material is supplied are 1,999 kPa and 558 kPa, respectively, the tissue repair material can not be discharged from a discharge part having an inner diameter smaller than that of the cylindrical container but can be discharged only from the opening portion having an inner diameter equal to the inner diameter of the cylindrical container (see the result of the 6.6 mm of the inner diameter of the discharge part, which is described as the reference value in Table 2).

The disclosure of JP2021-162131 filed on Sep. 30, 2021 is incorporated in the present specification by reference in its entirety. All documents, patent applications, and technical standards described in the present specification are herein incorporated by reference to the same extent that individual documents, patent applications, and technical standards have been specifically and individually indicated to be incorporated by reference, respectively.

### SEQUENCE LIST

International application based on the International Patent Cooperation Treaty 21F01232W1JP22029561_4.xml

What is claimed is:

1. A tissue repair material kit comprising:
a cylindrical container that has an opening portion having an opening diameter smaller than an inner diameter of the container; and
a tissue repair material,
wherein in a case where the tissue repair material is supplied, a storage elastic modulus of the tissue repair material at 25° C. is 0.1 kPa to 100 kPa.

2. The tissue repair material kit according to claim 1, further comprising:
a discharge part that has an inner diameter smaller than the inner diameter of the cylindrical container and is provided at the opening portion.

3. The tissue repair material kit according to claim 1,
wherein the opening diameter of the opening portion is 10% to 70% of the inner diameter of the cylindrical container.

4. The tissue repair material kit according to claim 2,
wherein the inner diameter of the discharge part is 10% to 70% of the inner diameter of the cylindrical container.

5. The tissue repair material kit according to claim 1,
wherein the opening diameter of the opening portion is 5 mm or less.

6. The tissue repair material kit according to claim 2,
wherein the inner diameter of the discharge part is 5 mm or less.

7. The tissue repair material kit according to claim 1,
wherein the tissue repair material has a particulate shape in a dry state, and
the opening diameter of the opening portion is 40% to 300% of a maximum particle diameter of the tissue repair material in the dry state.

8. The tissue repair material kit according to claim 2,
wherein the tissue repair material has a particulate shape in a dry state, and
the inner diameter of the discharge part is 40% to 300% of a maximum particle diameter of the tissue repair material in the dry state.

9. The tissue repair material kit according to claim 1, further comprising:
a pressurization unit,
wherein the opening portion is provided at one end of the cylindrical container, and
the pressurization is provided at the other end of the cylindrical container.

10. The tissue repair material kit according to claim 9,
wherein the pressurization unit is a plunger.

11. The tissue repair material kit according to claim 1,
wherein the tissue repair material is accommodated in the cylindrical container.

12. The tissue repair material kit according to claim 1,
wherein a water absorption rate of the tissue repair material is 300% to 2,000%.

13. The tissue repair material kit according to claim 1,
wherein in a case where the tissue repair material comprises a water-containing liquid, the storage elastic modulus of the tissue repair material at 25° C. is 0.1 kPa to 100 kPa in a case where a value obtained by multiplying a mass fraction of a content of the water-containing liquid with respect to a dry mass of the tissue repair material by a tap density of the tissue repair material in a dry state is 0.40 g/mL to 0.70 g/mL.

14. The tissue repair material kit according to claim 1, further comprising:
a water-containing liquid for mixing with the tissue repair material.

15. The tissue repair material kit according to claim 14,
wherein a content of the water-containing liquid is 50% by mass to 2,000% by mass with respect to a dry mass of the tissue repair material.

16. The tissue repair material kit according to claim 1,
wherein the tissue repair material comprises a biocompatible polymer.

17. The tissue repair material kit according to claim 16,
wherein the biocompatible polymer is a recombinant peptide.

18. The tissue repair material kit according to claim 17,
wherein the recombinant peptide comprises the following peptide of (A), (B), or (C):
(A) a peptide consisting of an amino acid sequence set forth in SEQ ID NO: 1;
(B) a peptide consisting of an amino acid sequence, in which one or several amino acid residues in the amino acid sequence set forth in SEQ ID NO: 1 are modified, and having biocompatibility; or
(C) a peptide consisting of an amino acid sequence which has a partial sequence having 80% or more sequence identity with a partial amino acid sequence consisting of 4th to 192nd amino acid residues in the amino acid sequence set forth in SEQ ID NO: 1, and having biocompatibility:
SEQ ID NO: 1:
GAP(GAPGLQGAPGLQGMPGERGAAGLPGPKGERGDAGPKGADGAPGAP
GLQGMPGERGAAGLPGPKGERGDAGPKGADGAPGKDGVRGLAGPIGPPG
ERGAAGLPGPKGERGDAGPKGADGAPGKDGVRGLAGPIGPPGPAGAPGA
PGLQGMPGERGAAGLPGPKGERGDAGPKGADGAPGKDGVRGLAGPP)
3G.

19. The tissue repair material kit according to claim 1,
wherein the tissue repair material is a bone tissue repair material.

20. A tissue repair method comprising:
a step of supplying the tissue repair material to a tissue-deficient part by using the tissue repair material kit according to claim 1.


SEQUENCE LISTING (10 sequences)

SEQ ID NO: 1 - protein, 571 aa, synthetic construct
  REGION 1..571: note = Synthetic: Recombinant
GAPGAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPGAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPGAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPG

SEQ ID NO: 2 - protein, 5 aa, synthetic construct
  REGION 1..5: note = Description of Artificial Sequence: adhesive sequence
YIGSR

SEQ ID NO: 3 - protein, 5 aa, synthetic construct
  REGION 1..5: note = Description of Artificial Sequence: adhesive sequence
PDSGR

SEQ ID NO: 4 - protein, 6 aa, synthetic construct
  REGION 1..6: note = Description of Artificial Sequence: adhesive sequence
LGTIPG

SEQ ID NO: 5 - protein, 5 aa, synthetic construct
  REGION 1..5: note = Description of Artificial Sequence: adhesive sequence
IKVAV

SEQ ID NO: 6 - protein, 4 aa, synthetic construct
  REGION 1..4: note = Description of Artificial Sequence: adhesive sequence
ERGD

SEQ ID NO: 7 - protein, 4 aa, synthetic construct
  REGION 1..4: note = Synthetic
DRGD

SEQ ID NO: 8 - protein, 3002 aa, synthetic construct
  REGION 1..3002: note = Synthetic
  VARIANT 1: note = X is any amino acid
  REPEAT 2..301: note = GXX repeats 3 to 100 times
  REPEAT 2..3001: note = (GXX)3-100 repeats 2 to 10 times
  VARIANT 3..4: note = X is any amino acid
  VARIANT 6..7: note = X is any amino acid
  VARIANT 9..10: note = X is any amino acid
  VARIANT 12..13: note = X is any amino acid
  VARIANT 15..16: note = X is any amino acid
  VARIANT 18..19: note = X is any amino acid
  VARIANT 21..22: note = X is any amino acid
  VARIANT 24..25: note = X is any amino acid
  VARIANT 27..28: note = X is any amino acid
  VARIANT 30..31: note = X is any amino acid
  VARIANT 33..34: note = X is any amino acid
  VARIANT 36..37: note = X is any amino acid
  VARIANT 39..40: note = X is any amino acid
  VARIANT 42..43: note = X is any amino acid
  VARIANT 45..46: note = X is any amino acid
  VARIANT 48..49: note = X is any amino acid
  VARIANT 51..52: note = X is any amino acid
  VARIANT 54..55: note = X is any amino acid
  VARIANT 57..58: note = X is any amino acid
  VARIANT 60..61: note = X is any amino acid
  VARIANT 63..64: note = X is any amino acid
  VARIANT 66..67: note = X is any amino acid
  VARIANT 69..70: note = X is any amino acid
  VARIANT 72..73: note = X is any amino acid
  VARIANT 75..76: note = X is any amino acid
  VARIANT 78..79: note = X is any amino acid
  VARIANT 81..82: note = X is any amino acid
  VARIANT 84..85: note = X is any amino acid
  VARIANT 87..88: note = X is any amino acid
  VARIANT 90..91: note = X is any amino acid
  VARIANT 93..94: note = X is any amino acid
  VARIANT 96..97: note = X is any amino acid
  VARIANT 99..100: note = X is any amino acid
  VARIANT 102..103: note = X is any amino acid
  VARIANT 105..106: note = X is any amino acid
  VARIANT 108..109: note = X is any amino acid
  VARIANT 111..112: note = X is any amino acid
  VARIANT 114..115: note = X is any amino acid
  VARIANT 117..118: note = X is any amino acid
  VARIANT 120..121: note = X is any amino acid
  VARIANT 123..124: note = X is any amino acid
  VARIANT 126..127: note = X is any amino acid
  VARIANT 129..130: note = X is any amino acid
  VARIANT 132..133: note = X is any amino acid
  VARIANT 135..136: note = X is any amino acid
  VARIANT 138..139: note = X is any amino acid
  VARIANT 141..142: note = X is any amino acid
  VARIANT 144..145: note = X is any amino acid
  VARIANT 147..148: note = X is any amino acid
  VARIANT 150..151: note = X is any amino acid
  VARIANT 153..154: note = X is any amino acid
  VARIANT 156..157: note = X is any amino acid
  VARIANT 159..160: note = X is any amino acid
  VARIANT 162..163: note = X is any amino acid
  VARIANT 165..166: note = X is any amino acid
  VARIANT 168..169: note = X is any amino acid
  VARIANT 171..172: note = X is any amino acid
  VARIANT 174..175: note = X is any amino acid
  VARIANT 177..178: note = X is any amino acid
  VARIANT 180..181: note = X is any amino acid
  VARIANT 183..184: note = X is any amino acid
  VARIANT 186..187: note = X is any amino acid
  VARIANT 189..190: note = X is any amino acid
  VARIANT 192..193: note = X is any amino acid
  VARIANT 195..196: note = X is any amino acid
  VARIANT 198..199: note = X is any amino acid
  VARIANT 201..202: note = X is any amino acid
  VARIANT 204..205: note = X is any amino acid
  VARIANT 207..208: note = X is any amino acid
  VARIANT 210..211: note = X is any amino acid
  VARIANT 213..214: note = X is any amino acid
  VARIANT 216..217: note = X is any amino acid
  VARIANT 219..220: note = X is any amino acid
  VARIANT 222..223: note = X is any amino acid
  VARIANT 225..226: note = X is any amino acid
  VARIANT 228..229: note = X is any amino acid
  VARIANT 231..232: note = X is any amino acid
  VARIANT 234..235: note = X is any amino acid
  VARIANT 237..238: note = X is any amino acid
  VARIANT 240..241: note = X is any amino acid
  VARIANT 243..244: note = X is any amino acid
  VARIANT 246..247: note = X is any amino acid
  VARIANT 249..250: note = X is any amino acid
  VARIANT 252..253: note = X is any amino acid
  VARIANT 255..256: note = X is any amino acid
  VARIANT 258..259: note = X is any amino acid
  VARIANT 261..262: note = X is any amino acid
  VARIANT 264..265: note = X is any amino acid
  VARIANT 267..268: note = X is any amino acid
  VARIANT 270..271: note = X is any amino acid
  VARIANT 273..274: note = X is any amino acid
  VARIANT 276..277: note = X is any amino acid
  VARIANT 279..280: note = X is any amino acid
  VARIANT 282..283: note = X is any amino acid
  VARIANT 285..286: note = X is any amino acid
  VARIANT 288..289: note = X is any amino acid
  VARIANT 291..292: note = X is any amino acid
  VARIANT 294..295: note = X is any amino acid
  VARIANT 297..298: note = X is any amino acid
  VARIANT 300..301: note = X is any amino acid
  VARIANT 303..304: note = X is any amino acid
  VARIANT 306..307: note = X is any amino acid
  VARIANT 309..310: note = X is any amino acid
  VARIANT 312..313: note = X is any amino acid
  VARIANT 315..316: note = X is any amino acid
  VARIANT 318..319: note = X is any amino acid
  VARIANT 321..322: note = X is any amino acid
  VARIANT 324..325: note = X is any amino acid
  VARIANT 327..328: note = X is any amino acid
  VARIANT 330..331: note = X is any amino acid
  VARIANT 333..334: note = X is any amino acid
  VARIANT 336..337: note = X is any amino acid
  VARIANT 339..340: note = X is any amino acid
  VARIANT 342..343: note = X is any amino acid
  VARIANT 345..346: note = X is any amino acid
  VARIANT 348..349: note = X is any amino acid
  VARIANT 351..352: note = X is any amino acid
  VARIANT 354..355: note = X is any amino acid
  VARIANT 357..358: note = X is any amino acid
  VARIANT 360..361: note = X is any amino acid
  VARIANT 363..364: note = X is any amino acid
  VARIANT 366..367: note = X is any amino acid
  VARIANT 369..370: note = X is any amino acid
  VARIANT 372..373: note = X is any amino acid
  VARIANT 375..376: note = X is any amino acid
  VARIANT 378..379: note = X is any amino acid
  VARIANT 381..382: note = X is any amino acid
  VARIANT 384..385: note = X is any amino acid
  VARIANT 387..388: note = X is any amino acid
  VARIANT 390..391: note = X is any amino acid
  VARIANT 393..394: note = X is any amino acid
  VARIANT 396..397: note = X is any amino acid
  VARIANT 399..400: note = X is any amino acid
  VARIANT 402..403: note = X is any amino acid
  VARIANT 405..406: note = X is any amino acid
  VARIANT 408..409: note = X is any amino acid
  VARIANT 411..412: note = X is any amino acid
  VARIANT 414..415: note = X is any amino acid
  VARIANT 417..418: note = X is any amino acid
  VARIANT 420..421: note = X is any amino acid
  VARIANT 423..424: note = X is any amino acid
  VARIANT 426..427: note = X is any amino acid
  VARIANT 429..430: note = X is any amino acid
  VARIANT 432..433: note = X is any amino acid
  VARIANT 435..436: note = X is any amino acid
  VARIANT 438..439: note = X is any amino acid
  VARIANT 441..442: note = X is any amino acid
  VARIANT 444..445: note = X is any amino acid
  VARIANT 447..448: note = X is any amino acid
  VARIANT 450..451: note = X is any amino acid
  VARIANT 453..454: note = X is any amino acid
  VARIANT 456..457: note = X is any amino acid
  VARIANT 459..460: note = X is any amino acid
  VARIANT 462..463: note = X is any amino acid
  VARIANT 465..466: note = X is any amino acid
  VARIANT 468..469: note = X is any amino acid
  VARIANT 471..472: note = X is any amino acid
  VARIANT 474..475: note = X is any amino acid
  VARIANT 477..478: note = X is any amino acid
  VARIANT 480..481: note = X is any amino acid
  VARIANT 483..484: note = X is any amino acid
  VARIANT 486..487: note = X is any amino acid
  VARIANT 489..490: note = X is any amino acid
  VARIANT 492..493: note = X is any amino acid
  VARIANT 495..496: note = X is any amino acid
  VARIANT 498..499: note = X is any amino acid
  VARIANT 501..502: note = X is any amino acid
  VARIANT 504..505: note = X is any amino acid
  VARIANT 507..508: note = X is any amino acid
  VARIANT 510..511: note = X is any amino acid
  VARIANT 513..514: note = X is any amino acid
  VARIANT 516..517: note = X is any amino acid
  VARIANT 519..520: note = X is any amino acid
  VARIANT 522..523: note = X is any amino acid
  VARIANT 525..526: note = X is any amino acid
  VARIANT 528..529: note = X is any amino acid
  VARIANT 531..532: note = X is any amino acid
  VARIANT 534..535: note = X is any amino acid
  VARIANT 537..538: note = X is any amino acid
  VARIANT 540..541: note = X is any amino acid
  VARIANT 543..544: note = X is any amino acid
  VARIANT 546..547: note = X is any amino acid
  VARIANT 549..550: note = X is any amino acid
  VARIANT 552..553: note = X is any amino acid
  VARIANT 555..556: note = X is any amino acid
  VARIANT 558..559: note = X is any amino acid
  VARIANT 561..562: note = X is any amino acid
  VARIANT 564..565: note = X is any amino acid
  VARIANT 567..568: note = X is any amino acid
  VARIANT 570..571: note = X is any amino acid
  VARIANT 573..574: note = X is any amino acid
  VARIANT 576..577: note = X is any amino acid
  VARIANT 579..580: note = X is any amino acid
  VARIANT 582..583: note = X is any amino acid
  VARIANT 585..586: note = X is any amino acid
  VARIANT 588..589: note = X is any amino acid
  VARIANT 591..592: note = X is any amino acid
  VARIANT 594..595: note = X is any amino acid
  VARIANT 597..598: note = X is any amino acid
  VARIANT 600..601: note = X is any amino acid
  VARIANT 603..604: note = X is any amino acid
  VARIANT 606..607: note = X is any amino acid
  VARIANT 609..610: note = X is any amino acid
  VARIANT 612..613: note = X is any amino acid
  VARIANT 615..616: note = X is any amino acid
  VARIANT 618..619: note = X is any amino acid
  VARIANT 621..622: note = X is any amino acid
  VARIANT 624..625: note = X is any amino acid
  VARIANT 627..628: note = X is any amino acid
  VARIANT 630..631: note = X is any amino acid
  VARIANT 633..634: note = X is any amino acid
  VARIANT 636..637: note = X is any amino acid
  VARIANT 639..640: note = X is any amino acid
  VARIANT 642..643: note = X is any amino acid
  VARIANT 645..646: note = X is any amino acid
  VARIANT 648..649: note = X is any amino acid
  VARIANT 651..652: note = X is any amino acid
  VARIANT 654..655: note = X is any amino acid
  VARIANT 657..658: note = X is any amino acid
  VARIANT 660..661: note = X is any amino acid
  VARIANT 663..664: note = X is any amino acid
  VARIANT 666..667: note = X is any amino acid
  VARIANT 669..670: note = X is any amino acid
  VARIANT 672..673: note = X is any amino acid
  VARIANT 675..676: note = X is any amino acid
  VARIANT 678..679: note = X is any amino acid
  VARIANT 681..682: note = X is any amino acid
  VARIANT 684..685: note = X is any amino acid
  VARIANT 687..688: note = X is any amino acid
  VARIANT 690..691: note = X is any amino acid
  VARIANT 693..694: note = X is any amino acid
  VARIANT 696..697: note = X is any amino acid
  VARIANT 699..700: note = X is any amino acid
  VARIANT 702..703: note = X is any amino acid
  VARIANT 705..706: note = X is any amino acid
  VARIANT 708..709: note = X is any amino acid
  VARIANT 711..712: note = X is any amino acid
  VARIANT 714..715: note = X is any amino acid
  VARIANT 717..718: note = X is any amino acid
  VARIANT 720..721: note = X is any amino acid
  VARIANT 723..724: note = X is any amino acid
  VARIANT 726..727: note = X is any amino acid
  VARIANT 729..730: note = X is any amino acid
  VARIANT 732..733: note = X is any amino acid
  VARIANT 735..736: note = X is any amino acid
  VARIANT 738..739: note = X is any amino acid
  VARIANT 741..742: note = X is any amino acid
  VARIANT 744..745: note = X is any amino acid
  VARIANT 747..748: note = X is any amino acid
  VARIANT 750..751: note = X is any amino acid
  VARIANT 753..754: note = X is any amino acid
  VARIANT 756..757: note = X is any amino acid
  VARIANT 759..760: note = X is any amino acid
  VARIANT 762..763: note = X is any amino acid
  VARIANT 765..766: note = X is any amino acid
  VARIANT 768..769: note = X is any amino acid
  VARIANT 771..772: note = X is any amino acid
  VARIANT 774..775: note = X is any amino acid
  VARIANT 777..778: note = X is any amino acid
  VARIANT 780..781: note = X is any amino acid
  VARIANT 783..784: note = X is any amino acid
  VARIANT 786..787: note = X is any amino acid
  VARIANT 789..790: note = X is any amino acid
  VARIANT 792..793: note = X is any amino acid
  VARIANT 795..796: note = X is any amino acid
  VARIANT 798..799: note = X is any amino acid
  VARIANT 801..802: note = X is any amino acid
  VARIANT 804..805: note = X is any amino acid
  VARIANT 807..808: note = X is any amino acid
  VARIANT 810..811: note = X is any amino acid
  VARIANT 813..814: note = X is any amino acid
  VARIANT 816..817: note = X is any amino acid
  VARIANT 819..820: note = X is any amino acid
  VARIANT 822..823: note = X is any amino acid
  VARIANT 825..826: note = X is any amino acid
  VARIANT 828..829: note = X is any amino acid
  VARIANT 831..832: note = X is any amino acid
  VARIANT 834..835: note = X is any amino acid
  VARIANT 837..838: note = X is any amino acid
  VARIANT 840..841: note = X is any amino acid
  VARIANT 843..844: note = X is any amino acid
  VARIANT 846..847: note = X is any amino acid
  VARIANT 849..850: note = X is any amino acid
  VARIANT 852..853: note = X is any amino acid
  VARIANT 855..856: note = X is any amino acid
  VARIANT 858..859: note = X is any amino acid
  VARIANT 861..862: note = X is any amino acid
  VARIANT 864..865: note = X is any amino acid
  VARIANT 867..868: note = X is any amino acid
  VARIANT 870..871: note = X is any amino acid
  VARIANT 873..874: note = X is any amino acid
  VARIANT 876..877: note = X is any amino acid
  VARIANT 879..880: note = X is any amino acid
  VARIANT 882..883: note = X is any amino acid
  VARIANT 885..886: note = X is any amino acid
  VARIANT 888..889: note = X is any amino acid
  VARIANT 891..892: note = X is any amino acid
  VARIANT 894..895: note = X is any amino acid
  VARIANT 897..898: note = X is any amino acid
  VARIANT 900..901: note = X is any amino acid
  VARIANT 903..904: note = X is any amino acid
  VARIANT 906..907: note = X is any amino acid
  VARIANT 909..910: note = X is any amino acid
  VARIANT 912..913: note = X is any amino acid
  VARIANT 915..916: note = X is any amino acid
  VARIANT 918..919: note = X is any amino acid
  VARIANT 921..922: note = X is any amino acid
  VARIANT 924..925: note = X is any amino acid
  VARIANT 927..928: note = X is any amino acid
  VARIANT 930..931: note = X is any amino acid
  VARIANT 933..934: note = X is any amino acid
  VARIANT 936..937: note = X is any amino acid
  VARIANT 939..940: note = X is any amino acid
  VARIANT 942..943: note = X is any amino acid
  VARIANT 945..946: note = X is any amino acid
  VARIANT 948..949: note = X is any amino acid
  VARIANT 951..952: note = X is any amino acid
  VARIANT 954..955: note = X is any amino acid
  VARIANT 957..958: note = X is any amino acid
  VARIANT 960..961: note = X is any amino acid
  VARIANT 963..964: note = X is any amino acid
  VARIANT 966..967: note = X is any amino acid
  VARIANT 969..970: note = X is any amino acid
  VARIANT 972..973: note = X is any amino acid
  VARIANT 975..976: note = X is any amino acid
  VARIANT 978..979: note = X is any amino acid
  VARIANT 981..982: note = X is any amino acid
  VARIANT 984..985: note = X is any amino acid
  VARIANT 987..988: note = X is any amino acid
  VARIANT 990..991: note = X is any amino acid
  VARIANT 993..994: note = X is any amino acid
  VARIANT 996..997: note = X is any amino acid
  VARIANT 999..1000: note = X is any amino acid
  VARIANT 1002..1003: note = X is any amino acid
  VARIANT 1005..1006: note = X is any amino acid
  VARIANT 1008..1009: note = X is any amino acid
  VARIANT 1011..1012: note = X is any amino acid
  VARIANT 1014..1015: note = X is any amino acid
  VARIANT 1017..1018: note = X is any amino acid
  VARIANT 1020..1021: note = X is any amino acid
  VARIANT 1023..1024: note = X is any amino acid
  VARIANT 1026..1027: note = X is any amino acid
  VARIANT 1029..1030: note = X is any amino acid
  VARIANT 1032..1033: note = X is any amino acid
  VARIANT 1035..1036: note = X is any amino acid
  VARIANT 1038..1039: note = X is any amino acid
  VARIANT 1041..1042: note = X is any amino acid
  VARIANT 1044..1045: note = X is any amino acid
  VARIANT 1047..1048: note = X is any amino acid
  VARIANT 1050..1051: note = X is any amino acid
  VARIANT 1053..1054: note = X is any amino acid
  VARIANT 1056..1057: note = X is any amino acid
  VARIANT 1059..1060: note = X is any amino acid
  VARIANT 1062..1063: note = X is any amino acid
  VARIANT 1065..1066: note = X is any amino acid
  VARIANT 1068..1069: note = X is any amino acid
  VARIANT 1071..1072: note = X is any amino acid
  VARIANT 1074..1075: note = X is any amino acid
  VARIANT 1077..1078: note = X is any amino acid
  VARIANT 1080..1081: note = X is any amino acid
  VARIANT 1083..1084: note = X is any amino acid
  VARIANT 1086..1087: note = X is any amino acid
  VARIANT 1089..1090: note = X is any amino acid
  VARIANT 1092..1093: note = X is any amino acid
  VARIANT 1095..1096: note = X is any amino acid
  VARIANT 1098..1099: note = X is any amino acid
  VARIANT 1101..1102: note = X is any amino acid
  VARIANT 1104..1105: note = X is any amino acid
  VARIANT 1107..1108: note = X is any amino acid
  VARIANT 1110..1111: note = X is any amino acid
  VARIANT 1113..1114: note = X is any amino acid
  VARIANT 1116..1117: note = X is any amino acid
  VARIANT 1119..1120: note = X is any amino acid
  VARIANT 1122..1123: note = X is any amino acid
  VARIANT 1125..1126: note = X is any amino acid
  VARIANT 1128..1129: note = X is any amino acid
  VARIANT 1131..1132: note = X is any amino acid
  VARIANT 1134..1135: note = X is any amino acid
  VARIANT 1137..1138: note = X is any amino acid
  VARIANT 1140..1141: note = X is any amino acid
  VARIANT 1143..1144: note = X is any amino acid
  VARIANT 1146..1147: note = X is any amino acid
  VARIANT 1149..1150: note = X is any amino acid
  VARIANT 1152..1153: note = X is any amino acid
  VARIANT 1155..1156: note = X is any amino acid
  VARIANT 1158..1159: note = X is any amino acid
  VARIANT 1161..1162: note = X is any amino acid
  VARIANT 1164..1165: note = X is any amino acid
  VARIANT 1167..1168: note = X is any amino acid
  VARIANT 1170..1171: note = X is any amino acid
  VARIANT 1173..1174: note = X is any amino acid
  VARIANT 1176..1177: note = X is any amino acid
  VARIANT 1179..1180: note = X is any amino acid
  VARIANT 1182..1183: note = X is any amino acid
  VARIANT 1185..1186: note = X is any amino acid
  VARIANT 1188..1189: note = X is any amino acid
  VARIANT 1191..1192: note = X is any amino acid
  VARIANT 1194..1195: note = X is any amino acid
  VARIANT 1197..1198: note = X is any amino acid
  VARIANT 1200..1201: note = X is any amino acid
  VARIANT 1203..1204: note = X is any amino acid
  VARIANT 1206..1207: note = X is any amino acid
  VARIANT 1209..1210: note = X is any amino acid
  VARIANT 1212..1213: note = X is any amino acid
  VARIANT 1215..1216: note = X is any amino acid
  VARIANT 1218..1219: note = X is any amino acid
  VARIANT 1221..1222: note = X is any amino acid
  VARIANT 1224..1225: note = X is any amino acid
  VARIANT 1227..1228: note = X is any amino acid
  VARIANT 1230..1231: note = X is any amino acid
  VARIANT 1233..1234: note = X is any amino acid
  VARIANT 1236..1237: note = X is any amino acid
  VARIANT 1239..1240: note = X is any amino acid
  VARIANT 1242..1243: note = X is any amino acid
  VARIANT 1245..1246: note = X is any amino acid
  VARIANT 1248..1249: note = X is any amino acid
  VARIANT 1251..1252: note = X is any amino acid
  VARIANT 1254..1255: note = X is any amino acid
  VARIANT 1257..1258: note = X is any amino acid
  VARIANT 1260..1261: note = X is any amino acid
  VARIANT 1263..1264: note = X is any amino acid
  VARIANT 1266..1267: note = X is any amino acid
  VARIANT 1269..1270: note = X is any amino acid
  VARIANT 1272..1273: note = X is any amino acid
  VARIANT 1275..1276: note = X is any amino acid
  VARIANT 1278..1279: note = X is any amino acid
  VARIANT 1281..1282: note = X is any amino acid
  VARIANT 1284..1285: note = X is any amino acid
  VARIANT 1287..1288: note = X is any amino acid
  VARIANT 1290..1291: note = X is any amino acid
  VARIANT 1293..1294: note = X is any amino acid
  VARIANT 1296..1297: note = X is any amino acid
  VARIANT 1299..1300: note = X is any amino acid
  VARIANT 1302..1303: note = X is any amino acid
  VARIANT 1305..1306: note = X is any amino acid
  VARIANT 1308..1309: note = X is any amino acid
  VARIANT 1311..1312: note = X is any amino acid
  VARIANT 1314..1315: note = X is any amino acid
  VARIANT 1317..1318: note = X is any amino acid
  VARIANT 1320..1321: note = X is any amino acid
  VARIANT 1323..1324: note = X is any amino acid
  VARIANT 1326..1327: note = X is any amino acid
  VARIANT 1329..1330: note = X is any amino acid
  VARIANT 1332..1333: note = X is any amino acid
  VARIANT 1335..1336: note = X is any amino acid
  VARIANT 1338..1339: note = X is any amino acid
  VARIANT 1341..1342: note = X is any amino acid
  VARIANT 1344..1345: note = X is any amino acid
  VARIANT 1347..1348: note = X is any amino acid
  VARIANT 1350..1351: note = X is any amino acid
  VARIANT 1353..1354: note = X is any amino acid
  VARIANT 1356..1357: note = X is any amino acid
  VARIANT 1359..1360: note = X is any amino acid
  VARIANT 1362..1363: note = X is any amino acid
  VARIANT 1365..1366: note = X is any amino acid
  VARIANT 1368..1369: note = X is any amino acid
  VARIANT 1371..1372: note = X is any amino acid
  VARIANT 1374..1375: note = X is any amino acid
  VARIANT 1377..1378: note = X is any amino acid
  VARIANT 1380..1381: note = X is any amino acid
  VARIANT 1383..1384: note = X is any amino acid
  VARIANT 1386..1387: note = X is any amino acid
  VARIANT 1389..1390: note = X is any amino acid
  VARIANT 1392..1393: note = X is any amino acid
  VARIANT 1395..1396: note = X is any amino acid
  VARIANT 1398..1399: note = X is any amino acid
  VARIANT 1401..1402: note = X is any amino acid
  VARIANT 1404..1405: note = X is any amino acid
  VARIANT 1407..1408: note = X is any amino acid
  VARIANT 1410..1411: note = X is any amino acid
  VARIANT 1413..1414: note = X is any amino acid
  VARIANT 1416..1417: note = X is any amino acid
  VARIANT 1419..1420: note = X is any amino acid
  VARIANT 1422..1423: note = X is any amino acid
  VARIANT 1425..1426: note = X is any amino acid
  VARIANT 1428..1429: note = X is any amino acid
  VARIANT 1431..1432: note = X is any amino acid
  VARIANT 1434..1435: note = X is any amino acid
  VARIANT 1437..1438: note = X is any amino acid
  VARIANT 1440..1441: note = X is any amino acid
  VARIANT 1443..1444: note = X is any amino acid
  VARIANT 1446..1447: note = X is any amino acid
  VARIANT 1449..1450: note = X is any amino acid
  VARIANT 1452..1453: note = X is any amino acid
  VARIANT 1455..1456: note = X is any amino acid
  VARIANT 1458..1459: note = X is any amino acid
  VARIANT 1461..1462: note = X is any amino acid
  VARIANT 1464..1465: note = X is any amino acid
  VARIANT 1467..1468: note = X is any amino acid
  VARIANT 1470..1471: note = X is any amino acid
  VARIANT 1473..1474: note = X is any amino acid
  VARIANT 1476..1477: note = X is any amino acid
  VARIANT 1479..1480: note = X is any amino acid
  VARIANT 1482..1483: note = X is any amino acid
  VARIANT 1485..1486: note = X is any amino acid
  VARIANT 1488..1489: note = X is any amino acid
  VARIANT 1491..1492: note = X is any amino acid
  VARIANT 1494..1495: note = X is any amino acid
  VARIANT 1497..1498: note = X is any amino acid
  VARIANT 1500..1501: note = X is any amino acid
  VARIANT 1503..1504: note = X is any amino acid
  VARIANT 1506..1507: note = X is any amino acid
  VARIANT 1509..1510: note = X is any amino acid
  VARIANT 1512..1513: note = X is any amino acid
  VARIANT 1515..1516: note = X is any amino acid
  VARIANT 1518..1519: note = X is any amino acid
  VARIANT 1521..1522: note = X is any amino acid
  VARIANT 1524..1525: note = X is any amino acid
  VARIANT 1527..1528: note = X is any amino acid
  VARIANT 1530..1531: note = X is any amino acid
  VARIANT 1533..1534: note = X is any amino acid
  VARIANT 1536..1537: note = X is any amino acid
  VARIANT 1539..1540: note = X is any amino acid
  VARIANT 1542..1543: note = X is any amino acid
  VARIANT 1545..1546: note = X is any amino acid
  VARIANT 1548..1549: note = X is any amino acid
  VARIANT 1551..1552: note = X is any amino acid
  VARIANT 1554..1555: note = X is any amino acid
  VARIANT 1557..1558: note = X is any amino acid
  VARIANT 1560..1561: note = X is any amino acid
  VARIANT 1563..1564: note = X is any amino acid
  VARIANT 1566..1567: note = X is any amino acid
  VARIANT 1569..1570: note = X is any amino acid
  VARIANT 1572..1573: note = X is any amino acid
  VARIANT 1575..1576: note = X is any amino acid
  VARIANT 1578..1579: note = X is any amino acid
  VARIANT 1581..1582: note = X is any amino acid
  VARIANT 1584..1585: note = X is any amino acid
  VARIANT 1587..1588: note = X is any amino acid
  VARIANT 1590..1591: note = X is any amino acid
  VARIANT 1593..1594: note = X is any amino acid
  VARIANT 1596..1597: note = X is any amino acid
  VARIANT 1599..1600: note = X is any amino acid
  VARIANT 1602..1603: note = X is any amino acid
  VARIANT 1605..1606: note = X is any amino acid
  VARIANT 1608..1609: note = X is any amino acid
  VARIANT 1611..1612: note = X is any amino acid
  VARIANT 1614..1615: note = X is any amino acid
  VARIANT 1617..1618: note = X is any amino acid
  VARIANT 1620..1621: note = X is any amino acid
  VARIANT 1623..1624: note = X is any amino acid
  VARIANT 1626..1627: note = X is any amino acid
  VARIANT 1629..1630: note = X is any amino acid
  VARIANT 1632..1633: note = X is any amino acid
  VARIANT 1635..1636: note = X is any amino acid
  VARIANT 1638..1639: note = X is any amino acid
  VARIANT 1641..1642: note = X is any amino acid
  VARIANT 1644..1645: note = X is any amino acid
  VARIANT 1647..1648: note = X is any amino acid
  VARIANT 1650..1651: note = X is any amino acid
  VARIANT 1653..1654: note = X is any amino acid
  VARIANT 1656..1657: note = X is any amino acid
  VARIANT 1659..1660: note = X is any amino acid
  VARIANT 1662..1663: note = X is any amino acid
  VARIANT 1665..1666: note = X is any amino acid
  VARIANT 1668..1669: note = X is any amino acid
  VARIANT 1671..1672: note = X is any amino acid
  VARIANT 1674..1675: note = X is any amino acid
  VARIANT 1677..1678: note = X is any amino acid
  VARIANT 1680..1681: note = X is any amino acid
  VARIANT 1683..1684: note = X is any amino acid
  VARIANT 1686..1687: note = X is any amino acid
  VARIANT 1689..1690: note = X is any amino acid
  VARIANT 1692..1693: note = X is any amino acid
  VARIANT 1695..1696: note = X is any amino acid
  VARIANT 1698..1699: note = X is any amino acid
  VARIANT 1701..1702: note = X is any amino acid
  VARIANT 1704..1705: note = X is any amino acid
  VARIANT 1707..1708: note = X is any amino acid
  VARIANT 1710..1711: note = X is any amino acid
  VARIANT 1713..1714: note = X is any amino acid
  VARIANT 1716..1717: note = X is any amino acid
  VARIANT 1719..1720: note = X is any amino acid
  VARIANT 1722..1723: note = X is any amino acid
  VARIANT 1725..1726: note = X is any amino acid
  VARIANT 1728..1729: note = X is any amino acid
  VARIANT 1731..1732: note = X is any amino acid
  VARIANT 1734..1735: note = X is any amino acid
  VARIANT 1737..1738: note = X is any amino acid
  VARIANT 1740..1741: note = X is any amino acid
  VARIANT 1743..1744: note = X is any amino acid
  VARIANT 1746..1747: note = X is any amino acid
  VARIANT 1749..1750: note = X is any amino acid
  VARIANT 1752..1753: note = X is any amino acid
  VARIANT 1755..1756: note = X is any amino acid
  VARIANT 1758..1759: note = X is any amino acid
  VARIANT 1761..1762: note = X is any amino acid
  VARIANT 1764..1765: note = X is any amino acid
  VARIANT 1767..1768: note = X is any amino acid
  VARIANT 1770..1771: note = X is any amino acid
  VARIANT 1773..1774: note = X is any amino acid
  VARIANT 1776..1777: note = X is any amino acid
  VARIANT 1779..1780: note = X is any amino acid
  VARIANT 1782..1783: note = X is any amino acid
  VARIANT 1785..1786: note = X is any amino acid
  VARIANT 1788..1789: note = X is any amino acid
  VARIANT 1791..1792: note = X is any amino acid
  VARIANT 1794..1795: note = X is any amino acid
  VARIANT 1797..1798: note = X is any amino acid
  VARIANT 1800..1801: note = X is any amino acid
  VARIANT 1803..1804: note = X is any amino acid
  VARIANT 1806..1807: note = X is any amino acid
  VARIANT 1809..1810: note = X is any amino acid
  VARIANT 1812..1813: note = X is any amino acid
  VARIANT 1815..1816: note = X is any amino acid
  VARIANT 1818..1819: note = X is any amino acid
  VARIANT 1821..1822: note = X is any amino acid
  VARIANT 1824..1825: note = X is any amino acid
  VARIANT 1827..1828: note = X is any amino acid
  VARIANT 1830..1831: note = X is any amino acid
  VARIANT 1833..1834: note = X is any amino acid
  VARIANT 1836..1837: note = X is any amino acid
  VARIANT 1839..1840: note = X is any amino acid
  VARIANT 1842..1843: note = X is any amino acid
  VARIANT 1845..1846: note = X is any amino acid
  VARIANT 1848..1849: note = X is any amino acid
  VARIANT 1851..1852: note = X is any amino acid
  VARIANT 1854..1855: note = X is any amino acid
  VARIANT 1857..1858: note = X is any amino acid
  VARIANT 1860..1861: note = X is any amino acid
  VARIANT 1863..1864: note = X is any amino acid
  VARIANT 1866..1867: note = X is any amino acid
  VARIANT 1869..1870: note = X is any amino acid
  VARIANT 1872..1873: note = X is any amino acid
  VARIANT 1875..1876: note = X is any amino acid
  VARIANT 1878..1879: note = X is any amino acid
  VARIANT 1881..1882: note = X is any amino acid
  VARIANT 1884..1885: note = X is any amino acid
  VARIANT 1887..1888: note = X is any amino acid
  VARIANT 1890..1891: note = X is any amino acid
  VARIANT 1893..1894: note = X is any amino acid
  VARIANT 1896..1897: note = X is any amino acid
  VARIANT 1899..1900: note = X is any amino acid
  VARIANT 1902..1903: note = X is any amino acid
  VARIANT 1905..1906: note = X is any amino acid
  VARIANT 1908..1909: note = X is any amino acid
  VARIANT 1911..1912: note = X is any amino acid
  VARIANT 1914..1915: note = X is any amino acid
  VARIANT 1917..1918: note = X is any amino acid
  VARIANT 1920..1921: note = X is any amino acid
  VARIANT 1923..1924: note = X is any amino acid
  VARIANT 1926..1927: note = X is any amino acid
  VARIANT 1929..1930: note = X is any amino acid
  VARIANT 1932..1933: note = X is any amino acid
  VARIANT 1935..1936: note = X is any amino acid
  VARIANT 1938..1939: note = X is any amino acid
  VARIANT 1941..1942: note = X is any amino acid
  VARIANT 1944..1945: note = X is any amino acid
  VARIANT 1947..1948: note = X is any amino acid
  VARIANT 1950..1951: note = X is any amino acid
  VARIANT 1953..1954: note = X is any amino acid
  VARIANT 1956..1957: note = X is any amino acid
  VARIANT 1959..1960: note = X is any amino acid
  VARIANT 1962..1963: note = X is any amino acid
  VARIANT 1965..1966: note = X is any amino acid
  VARIANT 1968..1969: note = X is any amino acid
  VARIANT 1971..1972: note = X is any amino acid
  VARIANT 1974..1975: note = X is any amino acid
  VARIANT 1977..1978: note = X is any amino acid
  VARIANT 1980..1981: note = X is any amino acid
  VARIANT 1983..1984: note = X is any amino acid
  VARIANT 1986..1987: note = X is any amino acid
  VARIANT 1989..1990: note = X is any amino acid
  VARIANT 1992..1993: note = X is any amino acid
  VARIANT 1995..1996: note = X is any amino acid
  VARIANT 1998..1999: note = X is any amino acid
  VARIANT 2001..2002: note = X is any amino acid
  VARIANT 2004..2005: note = X is any amino acid
  VARIANT 2007..2008: note = X is any amino acid
  VARIANT 2010..2011: note = X is any amino acid
  VARIANT 2013..2014: note = X is any amino acid
  VARIANT 2016..2017: note = X is any amino acid
  VARIANT 2019..2020: note = X is any amino acid
  VARIANT 2022..2023: note = X is any amino acid
  VARIANT 2025..2026: note = X is any amino acid
  VARIANT 2028..2029: note = X is any amino acid
  VARIANT 2031..2032: note = X is any amino acid
  VARIANT 2034..2035: note = X is any amino acid
  VARIANT 2037..2038: note = X is any amino acid
  VARIANT 2040..2041: note = X is any amino acid
  VARIANT 2043..2044: note = X is any amino acid
  VARIANT 2046..2047: note = X is any amino acid
  VARIANT 2049..2050: note = X is any amino acid
  VARIANT 2052..2053: note = X is any amino acid
  VARIANT 2055..2056: note = X is any amino acid
  VARIANT 2058..2059: note = X is any amino acid
  VARIANT 2061..2062: note = X is any amino acid
  VARIANT 2064..2065: note = X is any amino acid
  VARIANT 2067..2068: note = X is any amino acid
  VARIANT 2070..2071: note = X is any amino acid
  VARIANT 2073..2074: note = X is any amino acid
  VARIANT 2076..2077: note = X is any amino acid
  VARIANT 2079..2080: note = X is any amino acid
  VARIANT 2082..2083: note = X is any amino acid
  VARIANT 2085..2086: note = X is any amino acid
  VARIANT 2088..2089: note = X is any amino acid
  VARIANT 2091..2092: note = X is any amino acid
  VARIANT 2094..2095: note = X is any amino acid
  VARIANT 2097..2098: note = X is any amino acid
  VARIANT 2100..2101: note = X is any amino acid
  VARIANT 2103..2104: note = X is any amino acid
  VARIANT 2106..2107: note = X is any amino acid
  VARIANT 2109..2110: note = X is any amino acid
  VARIANT 2112..2113: note = X is any amino acid
  VARIANT 2115..2116: note = X is any amino acid
  VARIANT 2118..2119: note = X is any amino acid
  VARIANT 2121..2122: note = X is any amino acid
  VARIANT 2124..2125: note = X is any amino acid
  VARIANT 2127..2128: note = X is any amino acid
  VARIANT 2130..2131: note = X is any amino acid
  VARIANT 2133..2134: note = X is any amino acid
  VARIANT 2136..2137: note = X is any amino acid
  VARIANT 2139..2140: note = X is any amino acid
  VARIANT 2142..2143: note = X is any amino acid
  VARIANT 2145..2146: note = X is any amino acid
  VARIANT 2148..2149: note = X is any amino acid
  VARIANT 2151..2152: note = X is any amino acid
  VARIANT 2154..2155: note = X is any amino acid
  VARIANT 2157..2158: note = X is any amino acid
  VARIANT 2160..2161: note = X is any amino acid
  VARIANT 2163..2164: note = X is any amino acid
  VARIANT 2166..2167: note = X is any amino acid
  VARIANT 2169..2170: note = X is any amino acid
  VARIANT 2172..2173: note = X is any amino acid
  VARIANT 2175..2176: note = X is any amino acid
  VARIANT 2178..2179: note = X is any amino acid
  VARIANT 2181..2182: note = X is any amino acid
  VARIANT 2184..2185: note = X is any amino acid
  VARIANT 2187..2188: note = X is any amino acid
  VARIANT 2190..2191: note = X is any amino acid
  VARIANT 2193..2194: note = X is any amino acid
  VARIANT 2196..2197: note = X is any amino acid
  VARIANT 2199..2200: note = X is any amino acid
  VARIANT 2202..2203: note = X is any amino acid
  VARIANT 2205..2206: note = X is any amino acid
  VARIANT 2208..2209: note = X is any amino acid
  VARIANT 2211..2212: note = X is any amino acid
  VARIANT 2214..2215: note = X is any amino acid
  VARIANT 2217..2218: note = X is any amino acid
  VARIANT 2220..2221: note = X is any amino acid
  VARIANT 2223..2224: note = X is any amino acid
  VARIANT 2226..2227: note = X is any amino acid
  VARIANT 2229..2230: note = X is any amino acid
  VARIANT 2232..2233: note = X is any amino acid
  VARIANT 2235..2236: note = X is any amino acid
  VARIANT 2238..2239: note = X is any amino acid
  VARIANT 2241..2242: note = X is any amino acid
  VARIANT 2244..2245: note = X is any amino acid
  VARIANT 2247..2248: note = X is any amino acid
  VARIANT 2250..2251: note = X is any amino acid
  VARIANT 2253..2254: note = X is any amino acid
  VARIANT 2256..2257: note = X is any amino acid
  VARIANT 2259..2260: note = X is any amino acid
  VARIANT 2262..2263: note = X is any amino acid
  VARIANT 2265..2266: note = X is any amino acid
  VARIANT 2268..2269: note = X is any amino acid
  VARIANT 2271..2272: note = X is any amino acid
  VARIANT 2274..2275: note = X is any amino acid
  VARIANT 2277..2278: note = X is any amino acid
  VARIANT 2280..2281: note = X is any amino acid
  VARIANT 2283..2284: note = X is any amino acid
  VARIANT 2286..2287: note = X is any amino acid
  VARIANT 2289..2290: note = X is any amino acid
  VARIANT 2292..2293: note = X is any amino acid
  VARIANT 2295..2296: note = X is any amino acid
  VARIANT 2298..2299: note = X is any amino acid
  VARIANT 2301..2302: note = X is any amino acid
  VARIANT 2304..2305: note = X is any amino acid
  VARIANT 2307..2308: note = X is any amino acid
  VARIANT 2310..2311: note = X is any amino acid
  VARIANT 2313..2314: note = X is any amino acid
  VARIANT 2316..2317: note = X is any amino acid
  VARIANT 2319..2320: note = X is any amino acid
  VARIANT 2322..2323: note = X is any amino acid
  VARIANT 2325..2326: note = X is any amino acid
  VARIANT 2328..2329: note = X is any amino acid
  VARIANT 2331..2332: note = X is any amino acid
  VARIANT 2334..2335: note = X is any amino acid
  VARIANT 2337..2338: note = X is any amino acid
  VARIANT 2340..2341: note = X is any amino acid
  VARIANT 2343..2344: note = X is any amino acid
  VARIANT 2346..2347: note = X is any amino acid
  VARIANT 2349..2350: note = X is any amino acid
  VARIANT 2352..2353: note = X is any amino acid
  VARIANT 2355..2356: note = X is any amino acid
  VARIANT 2358..2359: note = X is any amino acid
  VARIANT 2361..2362: note = X is any amino acid
  VARIANT 2364..2365: note = X is any amino acid
  VARIANT 2367..2368: note = X is any amino acid
  VARIANT 2370..2371: note = X is any amino acid
  VARIANT 2373..2374: note = X is any amino acid
  VARIANT 2376..2377: note = X is any amino acid
  VARIANT 2379..2380: note = X is any amino acid
  VARIANT 2382..2383: note = X is any amino acid
  VARIANT 2385..2386: note = X is any amino acid
  VARIANT 2388..2389: note = X is any amino acid
  VARIANT 2391..2392: note = X is any amino acid
  VARIANT 2394..2395: note = X is any amino acid
  VARIANT 2397..2398: note = X is any amino acid
  VARIANT 2400..2401: note = X is any amino acid
  VARIANT 2403..2404: note = X is any amino acid
  VARIANT 2406..2407: note = X is any amino acid
  VARIANT 2409..2410: note = X is any amino acid
  VARIANT 2412..2413: note = X is any amino acid
  VARIANT 2415..2416: note = X is any amino acid
  VARIANT 2418..2419: note = X is any amino acid
  VARIANT 2421..2422: note = X is any amino acid
  VARIANT 2424..2425: note = X is any amino acid
  VARIANT 2427..2428: note = X is any amino acid
  VARIANT 2430..2431: note = X is any amino acid
  VARIANT 2433..2434: note = X is any amino acid
  VARIANT 2436..2437: note = X is any amino acid
  VARIANT 2439..2440: note = X is any amino acid
  VARIANT 2442..2443: note = X is any amino acid
  VARIANT 2445..2446: note = X is any amino acid
  VARIANT 2448..2449: note = X is any amino acid
  VARIANT 2451..2452: note = X is any amino acid
  VARIANT 2454..2455: note = X is any amino acid
  VARIANT 2457..2458: note = X is any amino acid
  VARIANT 2460..2461: note = X is any amino acid
  VARIANT 2463..2464: note = X is any amino acid
  VARIANT 2466..2467: note = X is any amino acid
  VARIANT 2469..2470: note = X is any amino acid
  VARIANT 2472..2473: note = X is any amino acid
  VARIANT 2475..2476: note = X is any amino acid
  VARIANT 2478..2479: note = X is any amino acid
  VARIANT 2481..2482: note = X is any amino acid
  VARIANT 2484..2485: note = X is any amino acid
  VARIANT 2487..2488: note = X is any amino acid
  VARIANT 2490..2491: note = X is any amino acid
  VARIANT 2493..2494: note = X is any amino acid
  VARIANT 2496..2497: note = X is any amino acid
  VARIANT 2499..2500: note = X is any amino acid
  VARIANT 2502..2503: note = X is any amino acid
  VARIANT 2505..2506: note = X is any amino acid
  VARIANT 2508..2509: note = X is any amino acid
  VARIANT 2511..2512: note = X is any amino acid
  VARIANT 2514..2515: note = X is any amino acid
  VARIANT 2517..2518: note = X is any amino acid
  VARIANT 2520..2521: note = X is any amino acid
  VARIANT 2523..2524: note = X is any amino acid
  VARIANT 2526..2527: note = X is any amino acid
  VARIANT 2529..2530: note = X is any amino acid
  VARIANT 2532..2533: note = X is any amino acid
  VARIANT 2535..2536: note = X is any amino acid
  VARIANT 2538..2539: note = X is any amino acid
  VARIANT 2541..2542: note = X is any amino acid
  VARIANT 2544..2545: note = X is any amino acid
  VARIANT 2547..2548: note = X is any amino acid
  VARIANT 2550..2551: note = X is any amino acid
  VARIANT 2553..2554: note = X is any amino acid
  VARIANT 2556..2557: note = X is any amino acid
  VARIANT 2559..2560: note = X is any amino acid
  VARIANT 2562..2563: note = X is any amino acid
  VARIANT 2565..2566: note = X is any amino acid
  VARIANT 2568..2569: note = X is any amino acid
  VARIANT 2571..2572: note = X is any amino acid
  VARIANT 2574..2575: note = X is any amino acid
  VARIANT 2577..2578: note = X is any amino acid
  VARIANT 2580..2581: note = X is any amino acid
  VARIANT 2583..2584: note = X is any amino acid
  VARIANT 2586..2587: note = X is any amino acid
  VARIANT 2589..2590: note = X is any amino acid
  VARIANT 2592..2593: note = X is any amino acid
  VARIANT 2595..2596: note = X is any amino acid
  VARIANT 2598..2599: note = X is any amino acid
  VARIANT 2601..2602: note = X is any amino acid
  VARIANT 2604..2605: note = X is any amino acid
  VARIANT 2607..2608: note = X is any amino acid
  VARIANT 2610..2611: note = X is any amino acid
  VARIANT 2613..2614: note = X is any amino acid
  VARIANT 2616..2617: note = X is any amino acid
  VARIANT 2619..2620: note = X is any amino acid
  VARIANT 2622..2623: note = X is any amino acid
  VARIANT 2625..2626: note = X is any amino acid
  VARIANT 2628..2629: note = X is any amino acid
  VARIANT 2631..2632: note = X is any amino acid
  VARIANT 2634..2635: note = X is any amino acid
  VARIANT 2637..2638: note = X is any amino acid
  VARIANT 2640..2641: note = X is any amino acid
  VARIANT 2643..2644: note = X is any amino acid
  VARIANT 2646..2647: note = X is any amino acid
  VARIANT 2649..2650: note = X is any amino acid
  VARIANT 2652..2653: note = X is any amino acid
  VARIANT 2655..2656: note = X is any amino acid
  VARIANT 2658..2659: note = X is any amino acid
  VARIANT 2661..2662: note = X is any amino acid
  VARIANT 2664..2665: note = X is any amino acid
  VARIANT 2667..2668: note = X is any amino acid
  VARIANT 2670..2671: note = X is any amino acid
  VARIANT 2673..2674: note = X is any amino acid
  VARIANT 2676..2677: note = X is any amino acid
  VARIANT 2679..2680: note = X is any amino acid
  VARIANT 2682..2683: note = X is any amino acid
  VARIANT 2685..2686: note = X is any amino acid
  VARIANT 2688..2689: note = X is any amino acid
  VARIANT 2691..2692: note = X is any amino acid
  VARIANT 2694..2695: note = X is any amino acid
  VARIANT 2697..2698: note = X is any amino acid
  VARIANT 2700..2701: note = X is any amino acid
  VARIANT 2703..2704: note = X is any amino acid
  VARIANT 2706..2707: note = X is any amino acid
  VARIANT 2709..2710: note = X is any amino acid
  VARIANT 2712..2713: note = X is any amino acid
  VARIANT 2715..2716: note = X is any amino acid
  VARIANT 2718..2719: note = X is any amino acid
  VARIANT 2721..2722: note = X is any amino acid
  VARIANT 2724..2725: note = X is any amino acid
  VARIANT 2727..2728: note = X is any amino acid
  VARIANT 2730..2731: note = X is any amino acid
  VARIANT 2733..2734: note = X is any amino acid
  VARIANT 2736..2737: note = X is any amino acid
  VARIANT 2739..2740: note = X is any amino acid
  VARIANT 2742..2743: note = X is any amino acid
  VARIANT 2745..2746: note = X is any amino acid
  VARIANT 2748..2749: note = X is any amino acid
  VARIANT 2751..2752: note = X is any amino acid
  VARIANT 2754..2755: note = X is any amino acid
  VARIANT 2757..2758: note = X is any amino acid
  VARIANT 2760..2761: note = X is any amino acid
  VARIANT 2763..2764: note = X is any amino acid
  VARIANT 2766..2767: note = X is any amino acid
  VARIANT 2769..2770: note = X is any amino acid
  VARIANT 2772..2773: note = X is any amino acid
  VARIANT 2775..2776: note = X is any amino acid
  VARIANT 2778..2779: note = X is any amino acid
  VARIANT 2781..2782: note = X is any amino acid
  VARIANT 2784..2785: note = X is any amino acid
  VARIANT 2787..2788: note = X is any amino acid
  VARIANT 2790..2791: note = X is any amino acid
  VARIANT 2793..2794: note = X is any amino acid
  VARIANT 2796..2797: note = X is any amino acid
  VARIANT 2799..2800: note = X is any amino acid
  VARIANT 2802..2803: note = X is any amino acid
  VARIANT 2805..2806: note = X is any amino acid
  VARIANT 2808..2809: note = X is any amino acid
  VARIANT 2811..2812: note = X is any amino acid
  VARIANT 2814..2815: note = X is any amino acid
  VARIANT 2817..2818: note = X is any amino acid
  VARIANT 2820..2821: note = X is any amino acid
  VARIANT 2823..2824: note = X is any amino acid
  VARIANT 2826..2827: note = X is any amino acid
  VARIANT 2829..2830: note = X is any amino acid
  VARIANT 2832..2833: note = X is any amino acid
  VARIANT 2835..2836: note = X is any amino acid
  VARIANT 2838..2839: note = X is any amino acid
  VARIANT 2841..2842: note = X is any amino acid
  VARIANT 2844..2845: note = X is any amino acid
  VARIANT 2847..2848: note = X is any amino acid
  VARIANT 2850..2851: note = X is any amino acid
  VARIANT 2853..2854: note = X is any amino acid
  VARIANT 2856..2857: note = X is any amino acid
  VARIANT 2859..2860: note = X is any amino acid
  VARIANT 2862..2863: note = X is any amino acid
  VARIANT 2865..2866: note = X is any amino acid
  VARIANT 2868..2869: note = X is any amino acid
  VARIANT 2871..2872: note = X is any amino acid
  VARIANT 2874..2875: note = X is any amino acid
  VARIANT 2877..2878: note = X is any amino acid
  VARIANT 2880..2881: note = X is any amino acid
  VARIANT 2883..2884: note = X is any amino acid
  VARIANT 2886..2887: note = X is any amino acid
  VARIANT 2889..2890: note = X is any amino acid
  VARIANT 2892..2893: note = X is any amino acid
  VARIANT 2895..2896: note = X is any amino acid
  VARIANT 2898..2899: note = X is any amino acid
  VARIANT 2901..2902: note = X is any amino acid
  VARIANT 2904..2905: note = X is any amino acid
  VARIANT 2907..2908: note = X is any amino acid
  VARIANT 2910..2911: note = X is any amino acid
  VARIANT 2913..2914: note = X is any amino acid
  VARIANT 2916..2917: note = X is any amino acid
  VARIANT 2919..2920: note = X is any amino acid
  VARIANT 2922..2923: note = X is any amino acid
  VARIANT 2925..2926: note = X is any amino acid
  VARIANT 2928..2929: note = X is any amino acid
  VARIANT 2931..2932: note = X is any amino acid
  VARIANT 2934..2935: note = X is any amino acid
  VARIANT 2937..2938: note = X is any amino acid
  VARIANT 2940..2941: note = X is any amino acid
  VARIANT 2943..2944: note = X is any amino acid
  VARIANT 2946..2947: note = X is any amino acid
  VARIANT 2949..2950: note = X is any amino acid
  VARIANT 2952..2953: note = X is any amino acid
  VARIANT 2955..2956: note = X is any amino acid
  VARIANT 2958..2959: note = X is any amino acid
  VARIANT 2961..2962: note = X is any amino acid
  VARIANT 2964..2965: note = X is any amino acid
  VARIANT 2967..2968: note = X is any amino acid
  VARIANT 2970..2971: note = X is any amino acid
  VARIANT 2973..2974: note = X is any amino acid
  VARIANT 2976..2977: note = X is any amino acid
  VARIANT 2979..2980: note = X is any amino acid
  VARIANT 2982..2983: note = X is any amino acid
  VARIANT 2985..2986: note = X is any amino acid
XGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXX

SEQ ID NO: 9 - protein, 571 aa, synthetic construct
  REGION 1..571: note = Synthetic
  VARIANT 5..6: note = X can be any naturally occurring amino acid
  VARIANT 8..9: note = X can be any naturally occurring amino acid
  VARIANT 11..12: note = X can be any naturally occurring amino acid
  VARIANT 14..15: note = X can be any naturally occurring amino acid
  VARIANT 17..18: note = X can be any naturally occurring amino acid
  VARIANT 20..21: note = X can be any naturally occurring amino acid
  VARIANT 23..24: note = X can be any naturally occurring amino acid
  VARIANT 26..27: note = X can be any naturally occurring amino acid
  VARIANT 29..30: note = X can be any naturally occurring amino acid
  VARIANT 32..33: note = X can be any naturally occurring amino acid
  VARIANT 35..36: note = X can be any naturally occurring amino acid
  VARIANT 38..39: note = X can be any naturally occurring amino acid
  VARIANT 41..42: note = X can be any naturally occurring amino acid
  VARIANT 44..45: note = X can be any naturally occurring amino acid
  VARIANT 47..48: note = X can be any naturally occurring amino acid
  VARIANT 50..51: note = X can be any naturally occurring amino acid
  VARIANT 53..54: note = X can be any naturally occurring amino acid
  VARIANT 56..57: note = X can be any naturally occurring amino acid
  VARIANT 59..60: note = X can be any naturally occurring amino acid
  VARIANT 62..63: note = X can be any naturally occurring amino acid
  VARIANT 65..66: note = X can be any naturally occurring amino acid
  VARIANT 68..69: note = X can be any naturally occurring amino acid
  VARIANT 71..72: note = X can be any naturally occurring amino acid
  VARIANT 74..75: note = X can be any naturally occurring amino acid
  VARIANT 77..78: note = X can be any naturally occurring amino acid
  VARIANT 80..81: note = X can be any naturally occurring amino acid
  VARIANT 83..84: note = X can be any naturally occurring amino acid
  VARIANT 86..87: note = X can be any naturally occurring amino acid
  VARIANT 89..90: note = X can be any naturally occurring amino acid
  VARIANT 92..93: note = X can be any naturally occurring amino acid
  VARIANT 95..96: note = X can be any naturally occurring amino acid
  VARIANT 98..99: note = X can be any naturally occurring amino acid
  VARIANT 101..102: note = X can be any naturally occurring amino acid
  VARIANT 104..105: note = X can be any naturally occurring amino acid
  VARIANT 107..108: note = X can be any naturally occurring amino acid
  VARIANT 110..111: note = X can be any naturally occurring amino acid
  VARIANT 113..114: note = X can be any naturally occurring amino acid
  VARIANT 116..117: note = X can be any naturally occurring amino acid
  VARIANT 119..120: note = X can be any naturally occurring amino acid
  VARIANT 122..123: note = X can be any naturally occurring amino acid
  VARIANT 125..126: note = X can be any naturally occurring amino acid
  VARIANT 128..129: note = X can be any naturally occurring amino acid
  VARIANT 131..132: note = X can be any naturally occurring amino acid
  VARIANT 134..135: note = X can be any naturally occurring amino acid
  VARIANT 137..138: note = X can be any naturally occurring amino acid
  VARIANT 140..141: note = X can be any naturally occurring amino acid
  VARIANT 143..144: note = X can be any naturally occurring amino acid
  VARIANT 146..147: note = X can be any naturally occurring amino acid
  VARIANT 149..150: note = X can be any naturally occurring amino acid
  VARIANT 152..153: note = X can be any naturally occurring amino acid
  VARIANT 155..156: note = X can be any naturally occurring amino acid
  VARIANT 158..159: note = X can be any naturally occurring amino acid
  VARIANT 161..162: note = X can be any naturally occurring amino acid
  VARIANT 164..165: note = X can be any naturally occurring amino acid
  VARIANT 167..168: note = X can be any naturally occurring amino acid
  VARIANT 170..171: note = X can be any naturally occurring amino acid
  VARIANT 173..174: note = X can be any naturally occurring amino acid
  VARIANT 176..177: note = X can be any naturally occurring amino acid
  VARIANT 179..180: note = X can be any naturally occurring amino acid
  VARIANT 182..183: note = X can be any naturally occurring amino acid
  VARIANT 185..186: note = X can be any naturally occurring amino acid
  VARIANT 188..189: note = X can be any naturally occurring amino acid
  VARIANT 191..192: note = X can be any naturally occurring amino acid
  VARIANT 194..195: note = X can be any naturally occurring amino acid
  VARIANT 197..198: note = X can be any naturally occurring amino acid
  VARIANT 200..201: note = X can be any naturally occurring amino acid
  VARIANT 203..204: note = X can be any naturally occurring amino acid
  VARIANT 206..207: note = X can be any naturally occurring amino acid
  VARIANT 209..210: note = X can be any naturally occurring amino acid
  VARIANT 212..213: note = X can be any naturally occurring amino acid
  VARIANT 215..216: note = X can be any naturally occurring amino acid
  VARIANT 218..219: note = X can be any naturally occurring amino acid
  VARIANT 221..222: note = X can be any naturally occurring amino acid
  VARIANT 224..225: note = X can be any naturally occurring amino acid
  VARIANT 227..228: note = X can be any naturally occurring amino acid
  VARIANT 230..231: note = X can be any naturally occurring amino acid
  VARIANT 233..234: note = X can be any naturally occurring amino acid
  VARIANT 236..237: note = X can be any naturally occurring amino acid
  VARIANT 239..240: note = X can be any naturally occurring amino acid
  VARIANT 242..243: note = X can be any naturally occurring amino acid
  VARIANT 245..246: note = X can be any naturally occurring amino acid
  VARIANT 248..249: note = X can be any naturally occurring amino acid
  VARIANT 251..252: note = X can be any naturally occurring amino acid
  VARIANT 254..255: note = X can be any naturally occurring amino acid
  VARIANT 257..258: note = X can be any naturally occurring amino acid
  VARIANT 260..261: note = X can be any naturally occurring amino acid
  VARIANT 263..264: note = X can be any naturally occurring amino acid
  VARIANT 266..267: note = X can be any naturally occurring amino acid
  VARIANT 269..270: note = X can be any naturally occurring amino acid
  VARIANT 272..273: note = X can be any naturally occurring amino acid
  VARIANT 275..276: note = X can be any naturally occurring amino acid
  VARIANT 278..279: note = X can be any naturally occurring amino acid
  VARIANT 281..282: note = X can be any naturally occurring amino acid
  VARIANT 284..285: note = X can be any naturally occurring amino acid
  VARIANT 287..288: note = X can be any naturally occurring amino acid
  VARIANT 290..291: note = X can be any naturally occurring amino acid
  VARIANT 293..294: note = X can be any naturally occurring amino acid
  VARIANT 296..297: note = X can be any naturally occurring amino acid
  VARIANT 299..300: note = X can be any naturally occurring amino acid
  VARIANT 302..303: note = X can be any naturally occurring amino acid
  VARIANT 305..306: note = X can be any naturally occurring amino acid
  VARIANT 308..309: note = X can be any naturally occurring amino acid
  VARIANT 311..312: note = X can be any naturally occurring amino acid
  VARIANT 314..315: note = X can be any naturally occurring amino acid
  VARIANT 317..318: note = X can be any naturally occurring amino acid
  VARIANT 320..321: note = X can be any naturally occurring amino acid
  VARIANT 323..324: note = X can be any naturally occurring amino acid
  VARIANT 326..327: note = X can be any naturally occurring amino acid
  VARIANT 329..330: note = X can be any naturally occurring amino acid
  VARIANT 332..333: note = X can be any naturally occurring amino acid
  VARIANT 335..336: note = X can be any naturally occurring amino acid
  VARIANT 338..339: note = X can be any naturally occurring amino acid
  VARIANT 341..342: note = X can be any naturally occurring amino acid
  VARIANT 344..345: note = X can be any naturally occurring amino acid
  VARIANT 347..348: note = X can be any naturally occurring amino acid
  VARIANT 350..351: note = X can be any naturally occurring amino acid
  VARIANT 353..354: note = X can be any naturally occurring amino acid
  VARIANT 356..357: note = X can be any naturally occurring amino acid
  VARIANT 359..360: note = X can be any naturally occurring amino acid
  VARIANT 362..363: note = X can be any naturally occurring amino acid
  VARIANT 365..366: note = X can be any naturally occurring amino acid
  VARIANT 368..369: note = X can be any naturally occurring amino acid
  VARIANT 371..372: note = X can be any naturally occurring amino acid
  VARIANT 374..375: note = X can be any naturally occurring amino acid
  VARIANT 377..378: note = X can be any naturally occurring amino acid
  VARIANT 380..381: note = X can be any naturally occurring amino acid
  VARIANT 383..384: note = X can be any naturally occurring amino acid
  VARIANT 386..387: note = X can be any naturally occurring amino acid
  VARIANT 389..390: note = X can be any naturally occurring amino acid
  VARIANT 392..393: note = X can be any naturally occurring amino acid
  VARIANT 395..396: note = X can be any naturally occurring amino acid
  VARIANT 398..399: note = X can be any naturally occurring amino acid
  VARIANT 401..402: note = X can be any naturally occurring amino acid
  VARIANT 404..405: note = X can be any naturally occurring amino acid
  VARIANT 407..408: note = X can be any naturally occurring amino acid
  VARIANT 410..411: note = X can be any naturally occurring amino acid
  VARIANT 413..414: note = X can be any naturally occurring amino acid
  VARIANT 416..417: note = X can be any naturally occurring amino acid
  VARIANT 419..420: note = X can be any naturally occurring amino acid
  VARIANT 422..423: note = X can be any naturally occurring amino acid
  VARIANT 425..426: note = X can be any naturally occurring amino acid
  VARIANT 428..429: note = X can be any naturally occurring amino acid
  VARIANT 431..432: note = X can be any naturally occurring amino acid
  VARIANT 434..435: note = X can be any naturally occurring amino acid
  VARIANT 437..438: note = X can be any naturally occurring amino acid
  VARIANT 440..441: note = X can be any naturally occurring amino acid
  VARIANT 443..444: note = X can be any naturally occurring amino acid
  VARIANT 446..447: note = X can be any naturally occurring amino acid
  VARIANT 449..450: note = X can be any naturally occurring amino acid
  VARIANT 452..453: note = X can be any naturally occurring amino acid
  VARIANT 455..456: note = X can be any naturally occurring amino acid
  VARIANT 458..459: note = X can be any naturally occurring amino acid
  VARIANT 461..462: note = X can be any naturally occurring amino acid
  VARIANT 464..465: note = X can be any naturally occurring amino acid
  VARIANT 467..468: note = X can be any naturally occurring amino acid
  VARIANT 470..471: note = X can be any naturally occurring amino acid
  VARIANT 473..474: note = X can be any naturally occurring amino acid
  VARIANT 476..477: note = X can be any naturally occurring amino acid
  VARIANT 479..480: note = X can be any naturally occurring amino acid
  VARIANT 482..483: note = X can be any naturally occurring amino acid
  VARIANT 485..486: note = X can be any naturally occurring amino acid
  VARIANT 488..489: note = X can be any naturally occurring amino acid
  VARIANT 491..492: note = X can be any naturally occurring amino acid
  VARIANT 494..495: note = X can be any naturally occurring amino acid
  VARIANT 497..498: note = X can be any naturally occurring amino acid
  VARIANT 500..501: note = X can be any naturally occurring amino acid
  VARIANT 503..504: note = X can be any naturally occurring amino acid
  VARIANT 506..507: note = X can be any naturally occurring amino acid
  VARIANT 509..510: note = X can be any naturally occurring amino acid
  VARIANT 512..513: note = X can be any naturally occurring amino acid
  VARIANT 515..516: note = X can be any naturally occurring amino acid
  VARIANT 518..519: note = X can be any naturally occurring amino acid
  VARIANT 521..522: note = X can be any naturally occurring amino acid
  VARIANT 524..525: note = X can be any naturally occurring amino acid
  VARIANT 527..528: note = X can be any naturally occurring amino acid
  VARIANT 530..531: note = X can be any naturally occurring amino acid
  VARIANT 533..534: note = X can be any naturally occurring amino acid
  VARIANT 536..537: note = X can be any naturally occurring amino acid
  VARIANT 539..540: note = X can be any naturally occurring amino acid
  VARIANT 542..543: note = X can be any naturally occurring amino acid
  VARIANT 545..546: note = X can be any naturally occurring amino acid
  VARIANT 548..549: note = X can be any naturally occurring amino acid
  VARIANT 551..552: note = X can be any naturally occurring amino acid
  VARIANT 554..555: note = X can be any naturally occurring amino acid
  VARIANT 557..558: note = X can be any naturally occurring amino acid
  VARIANT 560..561: note = X can be any naturally occurring amino acid
  VARIANT 563..564: note = X can be any naturally occurring amino acid
  VARIANT 566..567: note = X can be any naturally occurring amino acid
  VARIANT 569..570: note = X can be any naturally occurring amino acid
GAPGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXG

SEQ ID NO: 10 - protein, 189 aa, synthetic construct
  REGION 1..189: note = Synthetic
  VARIANT 2..3: note = X is any amino acid
  VARIANT 5..6: note = X is any amino acid
  VARIANT 8..9: note = X is any amino acid
  VARIANT 11..12: note = X is any amino acid
  VARIANT 14..15: note = X is any amino acid
  VARIANT 17..18: note = X is any amino acid
  VARIANT 20..21: note = X is any amino acid
  VARIANT 23..24: note = X is any amino acid
  VARIANT 26..27: note = X is any amino acid
  VARIANT 29..30: note = X is any amino acid
  VARIANT 32..33: note = X is any amino acid
  VARIANT 35..36: note = X is any amino acid
  VARIANT 38..39: note = X is any amino acid
  VARIANT 41..42: note = X is any amino acid
  VARIANT 44..45: note = X is any amino acid
  VARIANT 47..48: note = X is any amino acid
  VARIANT 50..51: note = X is any amino acid
  VARIANT 53..54: note = X is any amino acid
  VARIANT 56..57: note = X is any amino acid
  VARIANT 59..60: note = X is any amino acid
  VARIANT 62..63: note = X is any amino acid
  VARIANT 65..66: note = X is any amino acid
  VARIANT 68..69: note = X is any amino acid
  VARIANT 71..72: note = X is any amino acid
  VARIANT 74..75: note = X is any amino acid
  VARIANT 77..78: note = X is any amino acid
  VARIANT 80..81: note = X is any amino acid
  VARIANT 83..84: note = X is any amino acid
  VARIANT 86..87: note = X is any amino acid
  VARIANT 89..90: note = X is any amino acid
  VARIANT 92..93: note = X is any amino acid
  VARIANT 95..96: note = X is any amino acid
  VARIANT 98..99: note = X is any amino acid
  VARIANT 101..102: note = X is any amino acid
  VARIANT 104..105: note = X is any amino acid
  VARIANT 107..108: note = X is any amino acid
  VARIANT 110..111: note = X is any amino acid
  VARIANT 113..114: note = X is any amino acid
  VARIANT 116..117: note = X is any amino acid
  VARIANT 119..120: note = X is any amino acid
  VARIANT 122..123: note = X is any amino acid
  VARIANT 125..126: note = X is any amino acid
  VARIANT 128..129: note = X is any amino acid
  VARIANT 131..132: note = X is any amino acid
  VARIANT 134..135: note = X is any amino acid
  VARIANT 137..138: note = X is any amino acid
  VARIANT 140..141: note = X is any amino acid
  VARIANT 143..144: note = X is any amino acid
  VARIANT 146..147: note = X is any amino acid
  VARIANT 149..150: note = X is any amino acid
  VARIANT 152..153: note = X is any amino acid
  VARIANT 155..156: note = X is any amino acid
  VARIANT 158..159: note = X is any amino acid
  VARIANT 161..162: note = X is any amino acid
  VARIANT 164..165: note = X is any amino acid
  VARIANT 167..168: note = X is any amino acid
  VARIANT 170..171: note = X is any amino acid
  VARIANT 173..174: note = X is any amino acid
  VARIANT 176..177: note = X is any amino acid
  VARIANT 179..180: note = X is any amino acid
  VARIANT 182..183: note = X is any amino acid
  VARIANT 185..186: note = X is any amino acid
  VARIANT 188..189: note = X is any amino acid
GXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXXGXX